SEQUENCE LISTING (10 sequences)

SEQ ID NO: 1 - DNA, 27 bp, synthetic construct
  source 1..27: mol_type = other DNA
ttacatgtgactgtgttcagggtatag

SEQ ID NO: 2 - DNA, 25 bp, synthetic construct
  source 1..25: mol_type = other DNA
catagctagccgtacatccacatct

SEQ ID NO: 3 - DNA, 30 bp, synthetic construct
  source 1..30: mol_type = other DNA
ttagcggccgcgtacggctttagaaatgag

SEQ ID NO: 4 - DNA, 27 bp, synthetic construct
  source 1..27: mol_type = other DNA
ttcacgtagtgggtctgaactgggcac

SEQ ID NO: 5 - DNA, 22 bp, synthetic construct
  source 1..22: mol_type = other DNA
gtgctataacgcgactatctag

SEQ ID NO: 6 - DNA, 20 bp, synthetic construct
  source 1..20: mol_type = other DNA
tgttggtagttcttccgtgg

SEQ ID NO: 7 - DNA, 23 bp, synthetic construct
  source 1..23: mol_type = other DNA
tttggtaatggtttctcatgtgg

SEQ ID NO: 8 - DNA, 23 bp, synthetic construct
  source 1..23: mol_type = other DNA
gacatttagtttgagtgttcctg

SEQ ID NO: 9 - DNA, 24 bp, synthetic construct
  source 1..24: mol_type = other DNA
ctttcgcagcataagtagtatgtc

SEQ ID NO: 10 - DNA, 24 bp, synthetic construct
  source 1..24: mol_type = other DNA
cattaccgcttcattcttatattc

(19) United States
(12) Patent Application Publication
(10) Pub. No.: US 2026/0347795 A1
(43) Pub. Date: Nov. 5, 2026

(54) DIPLOID RECOMBINANT NUCLEOCYTOPLASMIC LARGE DNA VIRAL VECTORS

(71) Applicant: PROBIOGEN AG, Berlin (DE)

(72) Inventors: Ingo JORDAN, Berlin (DE); Volker SANDIG, Berlin (DE); Nicole SEEHASE, Rheinstetten (DE)

(21) Appl. No.: 19/485,650

(22) PCT Filed: May 13, 2024
(86) PCT No.: PCT/EP2024/063025

§ 371 Date: Nov. 18, 2025

(30) Foreign Application Priority Data
May 26, 2023 (EP) 23175635.4

(51) Int. Cl. C12N 7/02; A61K 35/76; C12N 5/07; C12N 5/071

(52) U.S. Cl. CPC C12N 7/025; A61K 35/76; C12N 5/0601; C12N 5/0602; C12N 2710/24021; C12N 2710/24052; C12N 2710/24121; C12N 2710/24152; C12N 2710/24221; C12N 2710/24252

(57) ABSTRACT

The present invention relates to a diploid recombinant nucleocytoplasmic large DNA virus (NCLDV) and uses thereof.

### CROSS REFERENCE TO RELATED APPLICATIONS

This application is a U.S. National Stage of PCT/EP2024/063025, international filing date May 13, 2024, which claims priority to European Application No. 23175635.4, filing date May 26, 2023.

The present invention relates to a diploid recombinant nucleocytoplasmic large DNA virus (NCLDV) and uses thereof.

### REFERENCE TO SEQUENCE LISTING IN ELECTRONIC FORMAT

The contents of the electronic sequence listing (513-44-pct-sequence-listing.xml; Size: 9,646 bytes; and Date of Creation: Nov. 13, 2025) is herein incorporated by reference in its entirety.

### BACKGROUND OF THE INVENTION

Viral vectors are derived from microorganisms that have evolved a high level of optimization for delivery and expression of compact genetic cassettes. However, the microorganisms the vectors are based upon are often pathogens that need to be attenuated before they can be used for vaccination or gene therapy. Several mechanisms are used in attenuation of viruses, including adaptation to replication below body temperature and to cellular substrates that are not encountered in the recipient. Attenuation of a vaccine vector via loss of virulence factors can also be advantageous for efficacy. The reason is that vaccine vectors should promote immune responses whereas pathogens aim for suppression of the involved physiological cascades. The disadvantage that may come with impaired defenses of the viral vector is an increased sensitivity to induction of innate immunity, a pathway that specifically interferes with robust antigen expression. Expression of transgenes is also impaired in highly attenuated vectors that do not replicate in vaccine recipients at all. This issue can be important because one parameter the immune system uses to discriminate acute and virulent pathogens against subacute or harmless microbes is an exponential expression pattern of foreign proteins and the accompanying damage to host cells.

Very high expression levels with an exponential start and a prolonged plateau phase are, therefore, important properties for successful immunomodulatory intervention. Such levels can be partially approached with viral promoters that have been designed to transcribe outside of the normal temporal regulation of the infectious cycle. Hybridization of early with late vaccinia promoters is one such example (Chakrabarti et al. 1997). However, a persistent limitation associated with insertion of highly expressing foreign genes is that disturbances are introduced into the genomic organization. Depending on the degree of expression level, microbial origin, and enzymatic activity of the transgene, any offending sequences are deleted within a few vector generations.

Recombinant poxviruses are important viral vectors for vaccination and oncotherapy that can accommodate large transgenes. Because poxviruses replicate without a nuclear phase, risk of chromosomal aberrations is low in case of an abortive infection. Risk of accidental interaction with wild-type viruses should be low because they are equipped to prevent superinfection. They furthermore replicate in viral factories that largely exclude defensive components of the host cell and material sourced from co-infecting viruses. Deletions of tandem repeats or non-essential regions, therefore, occur during rearrangements within the actively replicating genome, not by recombination across different genomes.

The viral genomic elements for replication are large Inverted Terminal Repeats (ITRs) that consist of 5-20 kb long stretches of homology at both ends of the viral DNA that are furthermore covalently sealed so that no 3′ or 5′ ends are exposed. While replication of the genome most likely initiates with hairpin mediated self-priming and strand-displacement, subsequent steps are not as clearly understood. Concatemers of full-length genomic segments may possibly be formed by rolling circle or discontinuous mechanisms. The concatemers are predicted to be separated by Holliday junctions that are dissolved into unit genomes by a viral endonuclease.

DNA replication fidelity is very high for double stranded (ds) DNA viruses. Because low mutation rates and high genomic stability interfere with adaptation to the defensive mechanisms of host organisms, nucleocytoplasmic large DNA viruses have established mutational hotspots to regain some degree of evolutionary flexibility. For poxviruses the hotspots are the telomers and for asfarviruses the multigene families dispersed across the viral genome. As a result, poxviral termini are destabilized by deletions and rearrangements under immunologic pressures. These rearrangements are known to interfere with viral replication.

For the Modified Vaccinia Ankara (MVA) virus, one of the highly attenuated poxviral vectors, the genetic changes that are discussed to be mainly responsible for the narrowed host range are the six deletions (deletion sites (DSs) I through VI). Deletion site (DS) III is commonly used as insertion site for transgenes (Kremer et al. 2012). DS I and IV are localized near the opposing viral telomers well outside of the region of homology. An MVA related virus is available where the right Inverted Terminal Repeat (ITR) has been replaced the left Inverted Terminal Repeat (ITR) (Jordan et al. 2019). This rearrangement has increased the size of homology at the two termini from 15 kbp to 27 kbp. Another consequence of this rearrangement is a loss of DS I and a duplication of DS IV. The two DS IV loci are positioned in the center of the two ITRs: the distance of DS IV to the proximal terminus is 11 kb, and the distance to the end of the repeat and start of the genome core is 16 kb.

Recombination of the viral genome with transgenes requires active genome replication. However, the above-described recombinant viruses have the disadvantage that they are not genetically stable enough and do not allow the desired high expression of heterologous genes.

Thus, there is an unmet need to improve and optimize these viruses as heterologous gene vehicles.

The present inventors provide a solution for the desired high expression of transgenes in nucleocytoplasmic large DNA viruses (NCLDV) such as poxviruses, including difficult genes, with a novel and surprising choice of design that also maintains genetic stability. They have inserted transgenes within the region of homology inside of the viral telomer of these viruses. The present inventors could show that this highly unusual insertion had important advantages. First, the diploid nature of the transgene increased copy stability via self-correction at the contralateral distal positions that surprisingly communicate across a distance of more than 200 kb. Second, the duplication of the expression cassette also duplicated the transgene doses which, in turn, increased transgene yield.

At the onset of this study, the present inventors have expected that the incorporation of transgenes into the Inverted Terminal Repeats (ITRs) will not be tolerated (because of indications in the literature that these may destabilize the replication signals) or will not be mechanistically possible (because the terminal repeats may be engaged in multi-strand helices and occupied by the replication machinery). The present inventors were surprised that it was indeed possible to stably embed transgenes within the homologous region of nucleocytoplasmic large DNA viruses (NCLDV). They were further surprised that the insertion of transgenes into a central site in an Inverted Terminal Repeat (ITR) did not interfere with the viability of nucleocytoplasmic large DNA viruses (NCLDV).

### SUMMARY OF THE INVENTION

In a first aspect, the present invention relates to a nucleocytoplasmic large DNA virus (NCLDV) containing a polynucleotide comprising an identical nucleotide sequence of interest within each Inverted Terminal Repeat (ITR).

In a second aspect, the present invention relates to a method for producing a nucleocytoplasmic large DNA virus (NCLDV) containing a polynucleotide comprising an identical nucleotide sequence of interest within each Inverted Terminal Repeat (ITR) comprising the steps of:
(i) transfecting a cell line infected with a nucleocytoplasmic large DNA virus (NCLDV) with a nucleotide sequence of interest, wherein the nucleotide sequence of interest is flanked by regions capable of homologous recombination within the Inverted Terminal Repeats (ITRs) of the NCLDV, thereby obtaining a NCLDV containing a polynucleotide comprising a nucleotide sequence of interest within one of the ITRs, and
(ii) culturing the NCLDV virus obtained in (i) under conditions allowing homologous repair between the Inverted Terminal Repeats (ITRs), thereby obtaining a NCLDV containing a polynucleotide comprising an identical nucleotide sequence of interest within each Inverted Terminal Repeat (ITR).

In a third aspect, the present invention relates to a nucleocytoplasmic large DNA virus (NCLDV) obtainable by the method according to the second aspect.

In a fourth aspect, the present invention relates to a method for propagating a nucleocytoplasmic large DNA virus (NCLDV) according to the first or third aspect comprising the step of:

culturing the NCLDV according to the first or third aspect in a cell line.

In a fifth aspect, the present invention relates to a pharmaceutical composition comprising a nucleocytoplasmic large DNA virus (NCLDV) according to the first or third aspect.

In a sixth aspect, the present invention relates to a nucleocytoplasmic large DNA virus (NCLDV) according to the first or third aspect, or a pharmaceutical composition according to the fifth aspect for use in medicine.

In a seventh aspect, the present invention relates to a nucleocytoplasmic large DNA virus (NCLDV) according to the first or third aspect, or a pharmaceutical composition according to the fifth aspect for use in therapy.

This summary of the invention does not describe all features of the invention.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The term “consisting essentially of”, as used herein, limits the scope of a claim to the specified materials or steps and those that do not materially affect the basic and novel characteristic(s) of the claimed invention. In other words, the term “consisting essentially of”, as used herein, is generally construed to mean that the composition or formulation (a) necessarily includes the listed ingredients and (b) is open to unlisted ingredients that do not materially affect the basic and novel properties of the composition. Similarly, when “consisting essentially of” is used herein in a process claim, the claim requires that the listed steps are performed, but also may include unlisted steps that do not affect the basic and material properties of the process.

The term “about” when used in connection with a numerical value is meant to encompass numerical values within a range having a lower limit that is 5% smaller than the indicated numerical value and having an upper limit that is 5% larger than the indicated numerical value.

The terms “polypeptide” and “protein” are used interchangeably in the context of the present invention and refer to a long peptide-linked chain of amino acids.

The term “virus”, as used herein, refers to a molecule that replicates only inside living cells of other organisms. It may also be cultivated in cell culture. Viruses can infect all types of life forms, from animals and plants to microorganisms including bacteria and archaea. While not inside an infected cell or in the process of infecting a cell, viruses exist in the form of independent particles. These viral particles, also known as virions, consist of two or three parts: (i) the genetic material made from either DNA or RNA, long nucleotides that carry genetic information, (ii) a protein coat, called the capsid, which surrounds and protects the genetic material, and in some cases (iii) an envelope of lipids that surrounds the protein coat when they are outside a cell. The shapes of these virus particles range from simple helical and icosahedral forms for some virus species to more complex structures for others. Thus, the term “virus”, as used herein, also encompasses viral particles.

The term “nucleocytoplasmic large DNA virus (NCLDV)”, as used herein, refers to a virus which belongs to a phylum of large DNA viruses with genomes ranging from 150 kb to 1.2 MB. The NCLDV infects a wide range of hosts including mammals, avian, arthropods, fish, and marine plankton. In addition, the NCLDV replicates either exclusively in the cytoplasm of the host cells or possess both cytoplasmic and nuclear stages in its life cycle. The NCLDV further comprises many genes involved in DNA repair, DNA replication, transcription, and translation. Typically, viruses with smaller genomes do not contain genes for these processes. There are nine families of NCLDVs that all share certain genomic and structural characteristics. However, it is uncertain whether the similarities of the different families of this group have a common viral ancestor. Specifically, the NCLDV phylum encompasses viruses of the Poxviridae family, an expansive viral family that includes major pathogens of humans and other mammals. The NCLDV phylum also includes members of the Asfarviridae and Iridoviridae family.

The Poxviridae are a diverse family with insects and vertebrates as hosts. They are large DNA viruses that replicate in the cytoplasm without a nuclear phase (a property that defines the phylum of Nucleocytoviricota and is shared with the Asfarviridae). The fully matured enveloped virions resemble flattened barrels with up to 300 nm in length and contain a genome of 128 to 365 kb that code for more than 200 genes. Poxviruses are studied as vectors for vaccination and oncotherapy due to a unique combination of virological properties. Attenuated poxviruses have been shown to be safe for recipients and able to rapidly induce a lasting and comprehensive immune response. Recombinant poxviral vectors can carry and stably maintain expression cassettes also for very large transgenes at different positions within the genome. Because poxviruses replicate without a nuclear phase risk of chromosomal aberrations is low in case of an abortive infection.

Some poxvirus species such as orf virus (genus Parapoxvirus) that naturally mainly infect sheep and goats have a narrow host range and can be further attenuated for use in humans with few genomic changes. Vaccinia virus (genus Orthopoxvirus) has a broad host range and strains such as Dairen I and Modified Vaccinia Ankara (MVA) virus were attenuated by serial passages in cell cultures that caused multiple mutations and deletions throughout the genome. The loss of certain combinations of pathogenicity factors not only reduced interference with host immunity (thus achieving attenuation) but may also have increased the beneficial immunogenic potential of some poxviral vectors.

Thus, more preferably, the virus of the Poxviridae family is selected from the group consisting of a vaccinia virus, an avipoxvirus, and a parapoxvirus. Even more preferably, the vaccinia virus is an attenuated vaccinia virus selected from the group consisting of a Modified Vaccinia Ankara (MVA) virus, an MVA related virus, a vaccinia virus Dairen-I, a vaccinia virus strain NYVAC, the avipoxvirus is selected from the group consisting of a canarypox virus and a fowlpox virus, or the parapoxvirus is an ORF virus.

The term “capsid”, as used herein, refers to the protein shell of a virus. It consists of several oligomeric (repeating) structural subunits made of proteins called protomers. The observable 3-dimensional morphological subunits, which may or may not correspond to individual proteins, are called capsomeres. The proteins making up the capsid of a virus are called “capsid proteins”. Alternatively, they are called “viral coat proteins (VCP)”.

The term “attenuated virus”, as used herein, refers to a virus with compromised virulence in the intended recipient, e.g. human or animal recipient. Such a property can be achieved by adaptation of a virus to narrow temperature ranges or narrow host ranges and to other artificial replication environments, including chemically defined media. Replication of such a virus is restricted in cells derived from the intended recipient, e.g. human or animal recipient, or in cells removed from the tissue environment. It may replicate to high titers outside of the intended recipient (e.g. in a permissive cell culture or laboratory animal). Specifically, Modified Vaccinia Ankara (MVA) viruses or MVA related viruses are attenuated viruses. They replicate in avian cells.

The term “highly attenuated virus”, as used herein, refers to a virus with blocked virulence in the intended recipient, e.g. human or animal recipient. Such a property can be achieved by adaptation of a virus to narrow temperature ranges or narrow host ranges and to other artificial replication environments, including chemically defined media. Replication of such a virus is blocked in cells derived from the intended recipient, e.g. human or animal recipient, or in cells removed from the tissue environment. It may replicate to high titers outside of the intended recipient (e.g. in a permissive cell/cell culture or laboratory animal). Specifically, the Modified Vaccinia Ankara (MVA) viruses or MVA related viruses can also be designated as highly attenuated viruses. They replicate in avian cells.

The term “host organism”, as used herein, refers to an organism which may be used for virus production and/or adaptation. The host organism may be a cell or an animal such as a laboratory animal. The cell may be an avian cell (e.g. a chicken, quail, goose, or duck cell such as a duck retina (CR) cell) or mammalian cell (e.g. human cell). The animal, particularly laboratory animal, may be a bird (e.g. a chicken, quail, goose, or duck), canine, mustela, rodent (e.g. a mouse, rat or hamster), an ovine, a caprine, pig, bat (e.g. a megabat or microbat) or a non-human primate (e.g. a monkey such as a great ape). Particularly, the vaccinia virus such as the MVA virus, the MVA related virus, the vaccinia virus Dairen-I, or the vaccinia virus strain NYVAC as well as the avipoxvirus such as the canarypox virus (ALVAC) or the fowlpox virus described herein replicate in an avian cell (e.g. in a chicken, quail, goose, or duck cell) or in a bird (e.g. in a chicken, quail, goose, or duck). Particularly, the parapoxvirus such as the ORF virus replicate in mammalian cells (e.g. human cells) or in a mammal (e.g. human).

The term “(host) cell”, as used herein, refers to a cell that is infected by a virus or another type of microorganism. For this virus or another type of microorganism, the host cell is a food source and/or a place to multiply or propagate.

Preferably, the (host) cell is an avian cell (e.g. in a chicken, quail, goose, or duck cell), a mammalian cell (e.g. human cell), an arthropod cell (e.g. mosquito cell), or a piscine cell line (e.g. gilt-head bream cell).

The term “(host) cell line”, as used herein, refers to a permanently established cell culture that proliferates indefinitely given appropriate fresh medium and space. A cell line typically arises from a primary cell culture. Primary cultures are initiated directly from the cells, tissues, or organs of animals and are typically used in experiments within a few days. By convention, the passaging or subcultivation of a primary culture begins a cell line. However, not all primary cultures yield cell lines. Instead, the cells of some subcultivated cultures die off slowly. The adaptation of cells to grow continuously in the laboratory is a very complex process who needs a lot of experience.

The term “non-adherent (host) cell”, as used herein, refers to a cell that is able to survive in a suspension culture without being attached to a surface (e.g. tissue culture plastic carrier or micro-carrier). Said cell may be a cell which can naturally live in suspension without being attached to a surface. Said cell may also be a cell which has been modified or adapted to be able to survive in a suspension culture without being attached to a surface (e.g. tissue culture plastic carrier or micro-carrier). As mentioned above, most cells are in their original, non-modified or non-adapted form, adherent cells. A non-adherent cell can usually be grown to a higher density than adherent conditions would allow. It is, thus, more suited for culturing in an industrial scale, e.g. in a bioreactor setting or in an agitated culture, for example, in order to produce proteins such as therapeutical proteins, e.g. antibodies or hormones. To make cells attractive for the production of recombinants, the cells have to be adapted to a non-adherent cell culture. Because the original cells would undergo apoptosis under serum-free conditions and/or in the absence of a suitable surface, this adaptation is a prolonged process requiring passaging with diminishing amounts of serum (e.g. dilution rows from 10% to 0% Fetal Calve Serum (FCS)), thereby selecting an irreversibly modified cell population. Adapted non-adherent cells are known in the art. The skilled person is aware of protocols for transferring a cell from an adherent state into a non-adherent state (see, for example, Appl Microbiol Biotechnol. 2008 March; 78(3):391-9. Epub 2008 Jan. 9). Cells comprised in a (host) cell line as described herein are usually non-adherent cells.

The term “serum-free conditions”, as used herein, refers to conditions, wherein cells grow in medium which is devoid of animal serum. Instead, cells grow in medium devoid of any animal derived components and preferably in a medium without any complex mixtures of biologic components, a so called “chemically defined medium”.

Preferably, the (host) cell line is selected from the group consisting of an avian cell line (preferably a chicken retina cell line AGE1.CR.PIX, a DF-1 chicken fibroblast cell line, or a duck embryonic stem cell line EB66), a mammalian cell line (preferably a baby hamster kidney (BHK) cell line or a human embryonal kidney 293 (HEK293) cell line), an arthropod cell line (preferably a Sf9 cell line), and a piscine cell line (preferably a SAF-1 cell line).

The term “virus passaging”, as used herein, refers to a process which involves infecting a series of host organisms, e.g. cells or animals such as laboratory animals, with a virus. Each time the virus is given some time to incubate, and then the next host organism is infected with the incubated virus. This process can also be designated as “serial virus passaging”. For example, serial virus passaging allows the generation of (highly) attenuated and/or host-restricted viruses. Specifically, Modified Vaccinia Ankara (MVA) viruses or MVA related viruses are (highly) attenuated viruses. They replicate in avian cells.

When a host organism, e.g. a cell/cell line such as an avian cell/cell line or an animal such as a laboratory animal, is defined by the term “permissive”, it refers to the fact that the virus is able to circumvent defenses of said organism and is able to invade a cell, replicate in said cell, and escape from said cell. Usually this occurs when the virus has modulated one or several of the cellular intrinsic defenses of said organism and/or the immune system of said organism.

The term “host-restricted virus”, as used herein, refers to a virus which (only or mainly) replicates in a specific host organism, e.g. in a cell such as an avian cell or in an animal such as a laboratory animal. It does not replicate or only replicates at very low levels in other organisms, e.g. in other cells than avian cells. A host-restricted virus may be achieved by (serial) virus passaging of a virus in a host organism, e.g. in avian cells. Specifically, Modified Vaccinia Ankara (MVA) viruses or MVA related viruses replicate in avian cells.

The term “infectious”, as used herein, refers to the ability of a virus to replicate in a cell and to produce viral particles. Infectivity can be evaluated either by detecting the virus load or by observing disease progression in a human or in an animal.

The term “isolated virus”, as used herein, refers to a virus that is removed from its native or culturing environment. Thus, an isolated virus may be free of some or all cellular components, i.e. components of the cells in which the virus naturally occurs or in which it is cultured (e.g. cytoplasmic or membrane components). It may also be free of some or all culturing components (e.g. culture medium or culture-related impurities such as culture-remnants).

The term “purified virus”, as used herein, refers to a virus that has been isolated under conditions that reduce or eliminate the presence of unrelated materials, i.e. contaminants, including native materials, e.g. cellular debris, cellular remnants, cellular proteins, cellular DNA molecules, and/or cellular RNA molecules, from which the virus is obtained. The purified virus is preferably substantially free of cell and/or culture components. As used herein, the term “substantially free” is used operationally, in the context of analytical testing of the material. A purified virus which is substantially free of contaminants is preferably at least 50% pure, more preferably at least 90% pure, and even more preferably at least 99% or 100% pure. Purity can be evaluated by chromatography, gel electrophoresis, immunoassay, composition analysis, biological assay, and other methods known in the art.

The term “Modified Vaccinia Ankara (MVA) virus”, as used herein, refers to a highly attenuated strain of vaccinia derived from the Ankara strain and developed for use as a vaccine and vaccine adjuvant. The original MVA virus was isolated from the wild-type Ankara strain by successive passage through chicken embryonic cells. Treated thus, it lost about 15% of the genome of wild-type vaccinia including its ability to replicate efficiently in primate (including human) cells.

The MVA virus contains a single copy of a double-stranded DNA genome, approximately 178 kb in length. The viral genomic DNA comprises a core region flanked by viral telomeres. In particular, the viral telomeres are located at the left and right site of the viral genomic DNA. Said telomeres further comprise Inverted Terminal Repeats (ITRs). The virus commonly accepted to be an MVA virus contains six characteristic deletion sites, called deletion sites I, II, III, IV, V, and VI. The numerals increase with the size of the deletion, not with position in the genome. The deletion site I is located in the left viral telomere, the deletion sites II, III, V, and VI are located in the core region. The deletion site IV is located in the right telomere. Only those viruses that contain all six deletion sites are considered bona fides MVA. The virus commonly accepted to be an MVA virus further comprises an (one) open reading frame for the (functional) gene products selected from the group consisting of C11R, C10L, D7L, A57R, B1R, B2R, B3R, B4R, B5R, B6R, B7R, B8R, B9R, B10R, B11R, B12R, B15R, B16R, B17L, B18R, B19R, and B22R. The method to describe such authentic/genuine MVAs has been published by Kremer et al., 2012. The MVA virus may have/comprise a sequence according to Genbank accession number U94848 (version U94848.1 and GI: 2772662).

The term “Modified Vaccinia Ankara (MVA) related virus”, as used herein, refers to a virus which structurally differs from the (above described or wild-type) MVA virus. It can, thus, not be considered to constitute a typical MVA virus anymore. It is rather a virus related to MVA. Said MVA related virus may, for example, not comprise a specific deletion site anymore, e.g. deletion site I, and/or comprise a specific deletion site in duplicate, e.g. deletion site IV. In addition, said MVA related virus may, for example, not comprise the open reading frame for a specific gene product anymore, e.g. the gene product C11, and/or comprise the open reading frame for a specific gene product in duplicate, e.g. gene product B1.

A specific example of an MVA related virus is a virus in whose genome/polynucleotide the right Inverted Terminal Repeat (ITR) has been replaced the left Inverted Terminal Repeat (ITR) (Jordan et al. 2019). This rearrangement has increased the size of homology at the two termini from 15 kbp to 27 kbp. Another consequence of this rearrangement is a loss of deletion site (DS) I and a duplication of DS IV. The two DS IV loci are positioned in the center of the two ITRs: the distance of DS IV to the proximal terminus is 11 kb, and the distance to the end of the repeat and start of the genome core is 16 kb. The MVA related virus may have/comprise a sequence according to Genbank Accession number KY633487 (version KY633487.1).

The term “viral telomeres”, as used herein, refers to sequences at the left and right site of the viral genomic DNA, e.g. of the MVA virus or MVA related virus. The sequence at the left site of the viral genomic DNA is designated as left viral telomere and the sequence at the right site of the viral genomic DNA is designated as right viral telomere. The viral telomeres contain Inverted Terminal Repeats (ITRs) that comprise or consist of regions of complementarity between the left and right sides of the genomic DNA. The viral telomeres also comprise functional and disrupted (not functional) genes that may be duplicated at both ends of the genomic DNA or that may be unique for the left or right side of the genomic DNA. Thus, viral telomeres extend beyond the mere region of the ITRs and can comprise 30 000 bp (30 kbp) or more.

The term “Inverted Terminal Repeats (ITRs)”, as used herein, refers to sequences comprising or consisting of regions of complementarity between the left and right sides of the viral genomic DNA, e.g. of the MVA virus or MVA related virus. With respect to the (wild-type) MVA virus, the ITR at the left side of the genomic DNA is designated as “left ITR” and the ITR at the right side of the genomic DNA is designated as “right ITR”. The MVA related virus contains, however, two copies of a nucleotide sequence comprising deletion site IV and the right ITR. Thus, with respect to the MVA related virus, it is in the following always referred to “the one” and “to the other” ITR.

The ITRs are part of the viral telomeres, in particular the left ITR is comprised in the left viral telomere and the right ITR is comprised in the right viral telomere. ITRs can form hairpin structures and appear to provide the origin for replication of the viral genomic DNA that presumably occurs by strand displacement of a leading strand and Okazaki-fragments in the lagging strand.

The term “terminus”, as used herein, refers to the left and right end of the viral genomic DNA, e.g. of the MVA virus or MVA related virus. The terminus is often covalently sealed so that no 3′ or 5′ end is exposed.

The term “core region”, as used herein, refers to a region of the viral genomic DNA, e.g. of the MVA virus or MVA related virus, comprising deletion sites V and III. In particular, the term “core region”, as used herein, refers to a region extending from deletion site V to deletion site III, wherein deletion sites V and III are included.

An “amino acid replacement” may also be designated herein as an “amino acid substitution”. The term “amino acid insertion”, as used herein in the context of a MVA related virus, refers to an amino acid modification which takes place within the amino acid sequence of the L3L, A3L, A34R, and/or A9L gene product(s), while the term “amino acid addition”, as used herein in the context of an MVA related virus, refers to an amino acid modification which takes place at the N- or C-terminus of the L3L, A3L, A34R, and/or A9L gene product(s).

Transgenes/nucleotide sequences of interest are often introduced into a site in the MVA virus that is naturally disrupted and, thus, does not contain essential factors. Deletion site (DS) III of the MVA virus is commonly used as insertion site for transgenes/nucleotide sequences of interest (Kremer et al. 2012). The present inventors recently described an MVA related virus where the right ITR has replaced the left ITR (see above). This rearrangement has increased the size of homology at the two termini from 15 kbp to 27 kbp and is accompanied by deletions and duplications of genes that may further modulate immunologic properties of the MVA related virus as a vaccine vector. Another consequence of this re-arrangement is a loss of DS I and duplication of DS IV. The two DS IV loci are positioned in the center of the two ITRs: the distance of DS IV to the proximal terminus is 11 kb, and the distance to the end of the repeat and start of the genome core is 16 kb. The present inventors have demonstrated that the insertion of transgenes/nucleotide sequences of interest into deletion site IV in the center of the ITR of the MVA related virus does not interfere with the viability of the virus. In addition, the MVA related virus is stable and, due to this insertion, transgene/nucleotide sequence of interest doses can be improved and, thereby, the transgene/nucleotide sequence of interest yield can be increased.

Residues in two or more polypeptides or polynucleotides are said to “correspond” to each other if the residues occupy an analogous position in the polypeptide or polynucleotide structures. It is well known in the art that analogous positions in two or more polypeptides or polynucleotides can be determined by aligning the polypeptide or polynucleotide sequences based on amino acid sequence or nucleotide sequence similarities. Such alignment tools are well known to the person skilled in the art and can be, for example, obtained on the World Wide Web, e.g. ClustalW (www.ebi.ac.uk/clustalw) or Align (http://www.ebi.ac.uk/emboss/align/index.html) using standard settings, preferably for Align EMBOSS: needle, Matrix: Blosum62, Gap Open 10.0, Gap Extend 0.5.

The term “productive replication”, as used herein, means, in the context of virus production, that more virus can be recovered at least once from an infected culture than virus that has been added to infect the culture. The virus may cause a cytopathic effect and replicates to levels that eventually result in massive cell death in the infected culture. As opposed to productive replication, reproductive replication can occur at very low levels without accompanying cytopathic effect and may eventually lead to loss of virus in a surviving culture.

The term “nucleotide sequence of interest”, as used herein, refers to a nucleotide sequence being of interest for the skilled person and/or whose expression product is of interest for the skilled person. Preferably, the nucleotide sequence of interest is selected from the group consisting of a sequence coding for an antigen, particularly an epitope of an antigen, a diagnostic compound, and a therapeutic compound. Specifically, the nucleotide sequence of interest is a heterologous nucleotide sequence.

The term “heterologous nucleotide sequence of interest”, as used herein, refers to a nucleotide sequence that is not normally found intimately associated with the NCLDV virus according to the present invention, in nature. NCLDV comprising a heterologous nucleotide sequence may also be designated as recombinant NCLDV. Preferably, the heterologous nucleotide sequence of interest is selected from the group consisting of a sequence coding for an antigen, particularly an epitope of an antigen, a diagnostic compound, and a therapeutic compound.

The term “homologous recombination”, as used herein, refers to a type of genetic recombination in which genetic information is exchanged between two similar or identical molecules of double-stranded or single-stranded nucleotides (usually DNA but may also be RNA). In the context of the present invention, the nucleotide sequence of interest is inserted into the ITRs of NCLDV, e.g. DS IV comprised in the ITRs of the MVA related virus, via homologous recombination.

Preferably, the nucleotide sequences of interest are stably maintained within the ITRs of the NCLDV. In the context of the present invention, the term “stably maintained” means that there are no deletions after at least 5, preferably after at least 10, more preferably after at least 20 virus passages, e.g. after at least 5, 6, 7, 8, 9, 10, 11, 12, 13, 14, 15, 16, 17, 18, 19, or 20 virus passages, and that the ITRs (still) contain the same nucleotide sequences of interest.

The term “homologous repair”, as used herein, refers to a process of homologous recombination which is carried out by viruses to restore complementarity of their Inverted Terminal Repeats (ITRs). This complementarity ensures the ability of the virus to replicate/propagate in host organisms and/or improves stability of the virus. The process of homologous repair can also be designated as self-correction or copy-correction or homology-directed repair (HDR).

The term “conditions allowing homologous repair”, as used herein, refers to conditions when there is a homologous region of DNA present in the viral genome or in the cytoplasm (e.g. provided by transfection of a shuttle plasmid). The conditions allowing homologous repair furthermore require replication of the viral genome and can thus occur only in a cell line that is permissive for an NCLDV according to the present invention. The conditions furthermore can include mechanisms that utilize single-strand annealing with or without involvement of a endonuclease or helicase.

The term “vaccine”, as used herein, refers to an agent that can be used to elicit protective immunity in a recipient, e.g. human or animal recipient. To be effective, a vaccine can elicit immunity in a portion of the immunized population, as some individuals may fail to mount a robust or protective immune response or, in some cases, any immune response. This inability may stem from the genetic background of the recipient or because of an immunodeficiency condition (either acquired or congenital) or immunosuppression (e.g., due to treatment with chemotherapy or use of immunosuppressive drugs).

The term “epitope (also known as antigenic determinant)”, as used herein, refers to the part of an antigen that is recognized by the immune system, specifically by antibodies, B cells, or T cells.

The term “vaccination”, as used herein, means that a recipient, e.g. human or animal recipient, is challenged with an infectious virus, e.g. in an attenuated or inactivated form of said infectious virus, to induce a specific immunity.

The term “treatment”, in particular “therapeutic treatment”, as used herein, refers to any therapy which improves the health status and/or prolongs (increases) the lifespan of a subject suffering from a disease. Said therapy may eliminate the disease in a subject, arrest or slow the development of the disease in a subject, inhibit the development of the disease in a subject, decrease the severity of symptoms in a subject suffering the disease, and/or decrease the recurrence in a subject who currently has or who previously has had a disease.

The term “pharmaceutically acceptable”, as used herein, means approved by a regulatory agency of the Federal or a state government or listed in the U.S. Pharmacopeia, European Pharmacopeia (Ph. Eur.) or other generally recognized pharmacopeia for use in animals, and more particularly in humans.

The term “excipient”, as used herein, is intended to indicate all substances in a pharmaceutical composition which are not active ingredients such as binders, lubricants, thickeners, surface active agents, preservatives, emulsifiers, buffers, flavoring agents, or colorants.

The term “diluent”, as used herein, relates to a diluting and/or thinning agent. Moreover, the term “diluent” includes a solution, suspension (e.g. liquid or solid suspension) and/or media.

The term “carrier”, as used herein, relates to one or more compatible solid or liquid fillers, which are suitable for an administration, e.g. to a human. The term “carrier” relates to a natural or synthetic organic or inorganic component which is combined with an active component in order to facilitate the application of the active component, or the permeation of the active component to the intended site of action. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds. A carrier compound that improves or facilitates permeation of the skin in topical applications is dimethyl sulfoxide (DMSO). Another preferred carrier consists of layered double hydroxide (LDH) nanoparticles. For example, such an LDH nanoparticles can be of the form [Mg3Al(OH)8](CH3CHOHCOO), that can be obtained, for example, by a reaction of Mg(lactate)₂ 3H₂O and Al (lactate) 3 in NaOH-controlled pH at 45-65 °C and protected from CO₂ and carbonic acids. A circularized RNA may intercalate into LDH nanoparticles and can be adminstered as LDH nanoparticle or be co-administered with VLPs.

Pharmaceutical carriers, diluents, and/or excipients can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilising agent(s). Examples of suitable binders include starch, gelatin, natural sugars such as glucose, lactose, sucrose, trehalose, corn sweeteners, natural and synthetic gums, such as acacia, tragacanth or sodium alginate, carboxymethyl cellulose, and polyethylene glycol. Examples of suitable lubricants include sodium oleate, sodium stearate, magnesium stearate, sodium benzoate, sodium acetate, sodium chloride, and the like. Preservatives, stabilizers, dyes, and even flavoring agents may be provided in the pharmaceutical composition. Examples of preservatives include sodium benzoate, sorbic acid, and esters of p-hydroxybenzoic acid. Antioxidants and suspending agents may be also used.

The term “subject”, as used herein, refers to any individual which may receive the virus as described herein. The term “subject”, as used herein, refers to any individual that/who may benefit from the treatment with the virus as described herein.

The subject may be a vertebrate, e.g. a human being, dog, cat, sheep, goat, cow, horse, camel or pig. It is particularly preferred that the “subject” is a human being.

The terms “subject”, “individual”, or “patient” are used interchangeably herein.

### EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

Recombination of the viral genome with transgenes requires active genome replication. However, the above-described recombinant viruses have the disadvantage that they are not genetically stable enough and do not allow the desired high expression of heterologous genes.

Thus, there is an unmet need to improve and optimize these viruses.

The present inventors provide a solution for the desired high expression of transgenes in nucleocytoplasmic large DNA viruses such as poxviruses, including difficult genes, with a novel and surprising choice of design that also maintains genetic stability. They have inserted transgenes within the region of homology inside of the viral telomer of these viruses. The present inventors could show that this highly unusual insertion had important advantages. First, the diploid nature of the transgene increased copy stability via self-correction at the contralateral distal positions that surprisingly communicate across a distance of more than 200 kb. Second, the duplication of the expression cassette also duplicated the transgene doses which, in turn, increased transgene yield.

At the onset of this study, the present inventors have expected that the incorporation of transgenes into the Inverted Terminal Repeats (ITRs) will not be tolerated (because of indications in the literature that these may destabilize the replication signals) or will not be mechanistically possible (because the terminal repeats may be engaged in multi-strand helices and occupied by the replication machinery). The present inventors were surprised that it was indeed possible to stably embed transgenes within the homologous region of nucleocytoplasmic large DNA viruses (NCLDV). They were further surprised that the insertion of transgenes into a central site in an Inverted Terminal Repeat (ITR) did not interfere with the viability of nucleocytoplasmic large DNA viruses (NCLDV).

Thus, in a first aspect, the present invention relates to a nucleocytoplasmic large DNA virus (NCLDV) containing a polynucleotide comprising an identical (heterologous) nucleotide sequence of interest within each Inverted Terminal Repeat (ITR).

Specifically, the (heterologous) nucleotide sequence of interest is comprised within each of the two Inverted Terminal Repeats (ITRs) contained in the NCLDV.

Thus, the (heterologous) nucleotide sequence of interest is comprised in diploid form in the NCLDV.

The polynucleotide contained within the NCLDV may represent the viral genome.

Preferably, the nucleotide sequence of interest is a heterologous/foreign nucleotide sequence of interest. The nucleotide sequence of interest can alternatively be designated as target nucleotide sequence.

More specifically, the identical (heterologous) nucleotide sequences of interest are comprised at identical positions, particularly mirror inverted identical positions within the Inverted Terminal Repeats (ITRs).

Thus, in one preferred embodiment, the present invention relates to a nucleocytoplasmic large DNA virus (NCLDV) containing a polynucleotide comprising an identical (heterologous) nucleotide sequence of interest within each (of the two) Inverted Terminal Repeat(s) (ITR(s)), wherein the identical (heterologous) nucleotide sequences of interest are comprised at mirror inverted identical positions within the (two) ITRs.

It will be understood that the identical (heterologous) nucleotide sequences of interest are preferably comprised between non-essential viral genes or replace one or more non-essential viral genes of the NCLDV. More preferably, the identical (heterologous) nucleotide sequences of interest are comprised, particularly between non-essential viral genes or replace one or more non-essential viral genes, in the center of the ITRs of the NCLDV.

Thus, in one more preferred embodiment, the present invention relates to a nucleocytoplasmic large DNA virus (NCLDV) containing a polynucleotide comprising an identical (heterologous) nucleotide sequence of interest within each (of the two) Inverted Terminal Repeat(s) (ITR(s)), wherein the identical (heterologous) nucleotide sequences of interest are comprised at mirror inverted identical positions, particularly between non-essential viral genes or replace one or more non-essential viral genes, within the center of the ITRs. The center of the ITRs can be characterized by a distance corresponding to half the width of the ITR +/−10% of the width, or +/−20% of the width, or +/−30% of the width.

Preferably, the NCLDV is a virus of the Poxviridae, Asfarviridae, or Iridoviridae family.

More preferably,
the virus of the Poxviridae family is selected from the group consisting of a vaccinia virus, an avipoxvirus, and a parapoxvirus,
the virus of the Asfarviridae family is an asfivirus, or
the virus of the Iridoviridae family is a lymphocystis disease virus (LCDV) or a Chloriridovirus.

Even more preferably,
the vaccinia virus is an attenuated vaccinia virus selected from the group consisting of a Modified Vaccinia Ankara (MVA) virus, an MVA related virus, a vaccinia virus Dairen-I, and
a vaccinia virus strain NYVAC,
the avipoxvirus is selected from the group consisting of a canarypox virus and a fowlpox virus, or
the parapoxvirus is an ORF virus.

In one even more preferred embodiment, the NCLDV is an MVA virus which comprises one of the identical (heterologous) nucleotide sequences of interest in the right Inverted Terminal Repeat (ITR) and the other one of the identical (heterologous) nucleotide sequences of interest in the left Inverted Terminal Repeat (ITR). Particularly, the identical (heterologous) nucleotide sequences of interest are comprised at mirror inverted identical positions within the ITRs. More particularly, the identical (heterologous) nucleotide sequences of interest are comprised at mirror inverted identical positions within the center of the ITRs. The center of the ITRs can be characterized by a distance corresponding to half the width of the ITR+/−10% of the width, or +/−20% of the width, or +/−30% of the width. For example, the center of the ITRs is 7.5+/−1.5 kbp from the sealed genomic termini for a wildtype MVA. The above-mentioned left ITR ranges, for example, from nucleotide position 166 to 9809 or from a nucleotide position corresponding thereto of the polynucleotide according to Genbank Accession number U94848 (version U94848.1 and GI: 2772662) and/or the above-mentioned right ITR ranges, for example, from nucleotide position 168280 to 177923 or from a nucleotide position corresponding thereto of the polynucleotide according to Genbank Accession number U94848 (version U94848.1 and GI: 2772662).

In one another even more preferred embodiment, the NCLDV is an MVA related virus which contains a polynucleotide comprising the identical (heterologous) nucleotide sequences of interest in each deletion site IV. Particularly, the nucleotide sequences of interest are comprised at mirror inverted identical positions within the (two) deletion sites IV. More particularly, the (two) deletion sites IV are positioned in the center of the ITRs. The center of the ITRs can be characterized by a distance corresponding to half the width of the ITR+/−10% of the width, or +/−20% of the width, or +/−30% of the width. For example, the center of the ITRs is 13.5 kbp+/−2.7 kbp for the MVA related virus (MVA-CR19). For the MVA related virus (MVA-CR19), the distance of DS IV to the proximal terminus is 11 kbp, and the distance to the end of the repeat and start of the genome core is 16 kbp. The ITRs range, for example, from nucleotide position 1 to 26920 or from a nucleotide position corresponding thereto of the polynucleotide according to Genbank Accession number KY633487 (version KY633487.1) and/or from nucleotide position 163630 to 190549 or from a nucleotide position corresponding thereto of the polynucleotide according to Genbank Accession number KY633487 (version KY633487.1).

Specifically, the MVA related virus contains a polynucleotide comprising two copies of a nucleotide sequence comprising deletion site IV and the right ITR and wherein one of the identical (heterologous) nucleotide sequences of interest is comprised in one copy of deletion site IV and the other one of the identical (heterologous) nucleotide sequences of interest is comprised in the other copy of deletion site IV.

Particularly, the nucleotide sequences of interest are comprised at mirror inverted identical positions within the (two) deletion sites IV. More particularly, the (two) deletion sites IV are positioned in the center of the ITRs. Especially, the distance of deletion site IV to the proximal terminus is 11 kb and the distance to the end of the repeat and start of the genome core is 16 kb. The one ITR ranges, for example, from nucleotide position 1 to 26920 or from a nucleotide position corresponding thereto of the polynucleotide according to Genbank Accession number KY633487 (version KY633487.1) and/or the other ITR ranges, for example, from nucleotide position 163630 to 190549 or from a nucleotide position corresponding thereto of the polynucleotide according to Genbank Accession number KY633487 (version KY633487.1).

More specifically, the Modified Vaccinia Ankara (MVA) related virus, as described herein, comprises (differs from (wild-type) MVA by) one or more, e.g. 1, 2, 3, 4, 5, 6, 7, or 8, of the following features:
(i) a nucleotide sequence corresponding to a region that includes the right Inverted Terminal Repeat (ITR) and extends to but excludes deletion site III instead of (replacing) a nucleotide sequence that includes the left Inverted Terminal Repeat (ITR) and extends to but excludes deletion site V,
(ii) two copies of a nucleotide sequence comprising deletion site IV and the right ITR,
(iii) no nucleotide sequence comprising deletion site I and the left ITR,
(iv) no deletion site I,
(v) two deletion sites IV,
(vi) no open reading frame for at least one gene product selected from the group consisting of C11R, C10L, and D7L,
(vii) two open reading frames for at least one gene product selected from the group consisting of A57R, BIR, B2R, B3R, B4R, B5R, B6R, B7R, B8R, B9R, BIOR, B11R, B12R, B15R, B16R, B17L, B18R, B19R, and B22R, and/or
(viii) a nucleotide sequence encoding a L3L gene product, wherein said nucleotide sequence comprises at least one mutation resulting in an amino acid sequence modification of said gene product.

The skilled person will be understood that the nucleotide sequences mentioned above are part of/comprised in the viral genomic DNA of the MVA related virus or are part of/comprised in the genome of the MVA related virus.

Even more specifically,
(i) the region that includes the right ITR and extends to but excludes deletion site III has a nucleotide sequence according Genbank Accession number KY633487 (preferably ranging from nucleotide position 162221 to 190549 or a nucleotide position corresponding thereto) or is a variant thereof which is at least 85%, at least 90%, at least 95% or 100%, e.g. at least 85, 86, 87, 88, 89, 90, 91, 92, 93, 94, 95, 96, 97, 98, 99% or 100%, identical to said nucleotide sequence,
(ii) the region that includes the left ITR and extends to but excludes deletion site V has a nucleotide sequence according to Genbank Accession number KY633487 (preferably ranging from nucleotide position 1 to 31261 or a nucleotide position corresponding thereto) or is a variant thereof which is at least 85%, at least 90%, at least 95% or 100%, e.g. at least 85, 86, 87, 88, 89, 90, 91, 92, 93, 94, 95, 96, 97, 98, 99% or 100%, identical to said nucleotide sequence, and/or
(iii) the nucleotide sequence comprising deletion site IV and the right ITR has a nucleotide sequence according to Genbank Accession number KY633487 (preferably ranging from nucleotide position 179272 to 190549 or a nucleotide position corresponding thereto) or is a variant thereof which is at least 85%, at least 90%, at least 95% or 100%, e.g. at least 85, 86, 87, 88, 89, 90, 91, 92, 93, 94, 95, 96, 97, 98, 99% or 100%, identical to said nucleotide sequence.

Still even more specifically, the MVA related virus (further) comprises a nucleotide sequence encoding a L3L gene product, an A3L gene product, an A34R gene product and an A9L gene product, wherein said nucleotide sequence comprise at least one mutation (e.g. 1, 2, 3, or 4 mutation(s)) resulting in an/at least one amino acid sequence modification (e.g. 1, 2, 3, or 4 amino acid modification(s)) of said gene products (i.e. said L3L gene product, said A3L gene product, said A34R gene product and said A9L gene product).

As to the MVA related virus and its modifications compared to the MVA virus, it is referred to WO 2018/153460 A1, specifically to pages 15 to 24 of the description of WO 2018/153460 A1. These pages are incorporated herein by reference.

Preferably, the nucleic acid sequences of interest are under control of/operably linked to a promoter. More preferably, the promoter is a NCLDV specific promoter. In case of a poxvirus, the NCLDV specific promoter may be a poxviral promoter. Even more preferably, the nucleotide sequences of interest are capable of being expressed from the NCLDV.

In one still even more preferred embodiment, the NCLDV is an MVA virus which comprises one of the identical (heterologous) nucleotide sequences of interest in the right Inverted Terminal Repeat (ITR) and the other one of the identical (heterologous) nucleotide sequences of interest in the left Inverted Terminal Repeat (ITR). The identical (heterologous) nucleotide sequences of interest are under control of/operably linked to a (poxvirus/MVA) virus specific promoter. The promoter allows expression of the identical (heterologous) nucleotide sequences of interest under suitable conditions. Particularly, the identical (heterologous) nucleotide sequences of interest are comprised at mirror inverted identical positions within the ITRs. More particularly, the identical (heterologous) nucleotide sequences of interest are comprised at mirror inverted identical positions within the center of the ITRs. The center of the ITRs can be characterized by a distance corresponding to half the width of the ITR+/−10% of the width, or +/−20% of the width, or +/−30% of the width. For example, the center of the ITRs is 7.5+/−1.5 kbp from the sealed genomic termini for a wildtype MVA. The above-mentioned left ITR ranges, for example, from nucleotide position 166 to 9809 or from a nucleotide position corresponding thereto of the polynucleotide according to Genbank Accession number U94848 (version U94848.1 and GI: 2772662) and/or the above-mentioned right ITR ranges, for example, from nucleotide position 168280 to 177923 or from a nucleotide position corresponding thereto of the polynucleotide according to Genbank Accession number U94848 (version U94848.1 and GI: 2772662).

In one another still even more preferred embodiment, the NCLDV is an MVA related virus which contains a polynucleotide comprising the identical (heterologous) nucleotide sequences of interest in each deletion site IV. The identical (heterologous) nucleotide sequences of interest are under control of/operably linked to a (poxvirus/MVA) virus specific promoter. The promoter allows expression of the identical (heterologous) nucleotide sequences of interest under suitable conditions. Particularly, the nucleotide sequences of interest are comprised at mirror inverted identical positions within the (two) deletion sites IV. More particularly, the (two) deletion sites IV are positioned in the center of the ITRs. The center of the ITRs can be characterized by a distance corresponding to half the width of the ITR+/−10% of the width, or +/−20% of the width, or +/−30% of the width. For example, the center of the ITRs is 13.5 kbp+/−2.7 kbp for the MVA related virus (MVA-CR19). For the MVA related virus (MVA-CR19), the distance of DS IV to the proximal terminus is 11 kbp, and the distance to the end of the repeat and start of the genome core is 16 kbp. The ITRs range, for example, from nucleotide position 1 to 26920 or from a nucleotide position corresponding thereto of the polynucleotide according to Genbank Accession number KY633487 (version KY633487.1) and/or from nucleotide position 163630 to 190549 or from a nucleotide position corresponding thereto of the polynucleotide according to Genbank Accession number KY633487 (version KY633487.1).

Specifically, the MVA related virus contains a polynucleotide comprising two copies of a nucleotide sequence comprising deletion site IV and the right ITR and wherein one of the identical (heterologous) nucleotide sequences of interest is comprised in one copy of deletion site IV and the other one of the identical (heterologous) nucleotide sequences of interest is comprised in the other copy of deletion site IV.

Particularly, the nucleotide sequences of interest are comprised at mirror inverted identical positions within the deletion sites IV. More particularly, the deletion sites IV are positioned in the center of the two ITRs. Especially, the distance of deletion site IV to the proximal terminus is 11 kb and the distance to the end of the repeat and start of the genome core is 16 kb. The one ITR ranges, for example, from nucleotide position 1 to 26920 or from a nucleotide position corresponding thereto of the polynucleotide according to Genbank Accession number KY633487 (version KY633487.1) and/or the other ITR ranges, for example, from nucleotide position 163630 to 190549 or from a nucleotide position corresponding thereto of the polynucleotide according to Genbank Accession number KY633487 (version KY633487.1).

Preferably, the nucleotide sequences of interest are stably maintained by/within the NCLDV. More particularly, the nucleotide sequences of interest are stably maintained by/within the NCLDV so that no deletions occur after at least 5, preferably after at least 10, more preferably after at least 20 virus passages, e.g. after at least 5, 6, 7, 8, 9, 10, 11, 12, 13, 14, 15, 16, 17, 18, 19, or 20 virus passages.

In one most preferred embodiment, the NCLDV is an MVA virus which stably comprises one of the identical (heterologous) nucleotide sequences of interest in the right Inverted Terminal Repeat (ITR) and the other one of the identical (heterologous) nucleotide sequences of interest in the left Inverted Terminal Repeat (ITR). The identical (heterologous) nucleotide sequences of interest are under control of/operably linked to a (poxvirus/MVA) virus specific promoter. The promoter allows expression of the identical (heterologous) nucleotide sequences of interest under suitable conditions. Particularly, the identical (heterologous) nucleotide sequences of interest are comprised at mirror inverted identical positions within the ITRs. More particularly, the identical (heterologous) nucleotide sequences of interest are comprised at mirror inverted identical positions within the center of the ITRs. The center of the ITRs can be characterized by a distance corresponding to half the width of the ITR+/−10% of the width, or +/−20% of the width, or +/−30% of the width. For example, the center of the ITRs is 7.5+/−1.5 kbp from the sealed genomic termini for a wildtype MVA. The above-mentioned left ITR ranges, for example, from nucleotide position 166 to 9809 or from a nucleotide position corresponding thereto of the polynucleotide according to Genbank Accession number U94848 (version U94848.1 and GI: 2772662) and/or the above-mentioned right ITR ranges, for example, from nucleotide position 168280 to 177923 or from a nucleotide position corresponding thereto of the polynucleotide according to Genbank Accession number U94848 (version U94848.1 and GI: 2772662).

In one another most preferred embodiment, the NCLDV is an MVA related virus which contains a polynucleotide stably comprising the identical (heterologous) nucleotide sequences of interest in each deletion site IV. The identical (heterologous) nucleotide sequences of interest are under control of/operably linked to a (poxvirus/MVA) virus specific promoter. The promoter allows expression of the identical (heterologous) nucleotide sequences of interest under suitable conditions. Particularly, the nucleotide sequences of interest are comprised at mirror inverted identical positions within the (two) deletion sites IV. More particularly, the (two) deletion sites IV are positioned in the center of the ITRs. The center of the ITRs can be characterized by a distance corresponding to half the width of the ITR+/−10% of the width, or +/−20% of the width, or +/−30% of the width. For example, the center of the ITRs is 13.5 kbp+/−2.7 kbp for the MVA related virus (MVA-CR19). For the MVA related virus (MVA-CR19), the distance of DS IV to the proximal terminus is 11 kbp, and the distance to the end of the repeat and start of the genome core is 16 kbp. The ITRs range, for example, from nucleotide position 1 to 26920 or from a nucleotide position corresponding thereto of the polynucleotide according to Genbank Accession number KY633487 (version KY633487.1) and/or from nucleotide position 163630 to 190549 or from a nucleotide position corresponding thereto of the polynucleotide according to Genbank Accession number KY633487 (version KY633487.1).

Specifically, the MVA related virus contains a polynucleotide comprising two copies of a nucleotide sequence comprising deletion site IV and the right ITR and wherein one of the identical (heterologous) nucleotide sequences of interest is stably comprised in one copy of deletion site IV and the other one of the identical (heterologous) nucleotide sequences of interest is stably comprised in the other copy of deletion site IV.

The one ITR ranges, for example, from nucleotide position 1 to 26920 or from a nucleotide position corresponding thereto of the polynucleotide according to Genbank Accession number KY633487 (version KY633487.1) and/or the other ITR ranges, for example, from nucleotide position 163630 to 190549 or from a nucleotide position corresponding thereto of the polynucleotide according to Genbank Accession number KY633487 (version KY633487.1).

The suitable conditions allowing expression of the identical (heterologous) nucleotide sequences of interest in the cell line can be described as follows: The identical (heterologous) nucleotide sequences of interest comprise a viral promoter that is operationally linked (followed by) an open reading frame for a gene product of interest. Infection of a cell or a cell line leads to transcription of an mRNA encoding the gene product of interest. Expression (transcription and translation) occurs both in a productive infection (where virus replicates) or a non-productive infection (where no progeny viruses are formed but initial steps of the infectious cycle are performed).

Preferably, the (heterologous) nucleotide sequences of interest are selected from the group consisting of a sequence coding for an antigen, particularly an epitope of an antigen, a diagnostic compound, and a therapeutic compound. More preferably, the therapeutic compound is a virotherapeutic compound, a vaccine, or an oncolytic compound.

In a second aspect, the present invention relates to a method for producing a nucleocytoplasmic large DNA virus (NCLDV) containing a polynucleotide comprising an identical (heterologous) nucleotide sequence of interest within each (of the two) Inverted Terminal Repeat(s) (ITR(s)) comprising the steps of:
(i) transfecting a cell line infected with a nucleocytoplasmic large DNA virus (NCLDV) with a (heterologous) nucleotide sequence of interest, wherein the (heterologous) nucleotide sequence of interest is flanked by regions capable of homologous recombination within the Inverted Terminal Repeats (ITRs) of the NCLDV, thereby obtaining a NCLDV containing a polynucleotide comprising a (heterologous) nucleotide sequence of interest within one of the ITRs, and
(ii) culturing the NCLDV virus obtained in (i) under conditions allowing homologous repair between the Inverted Terminal Repeats (ITRs), thereby obtaining a NCLDV containing a polynucleotide comprising an identical (heterologous) nucleotide sequence of interest within each (of the two) Inverted Terminal Repeat(s) (ITR(s)).

The polynucleotide contained within the NCLDV may represent the viral genome.

Preferably, the nucleotide sequence of interest is a heterologous/foreign nucleotide sequence of interest. The nucleotide sequence of interest can alternatively be designated as target nucleotide sequence.

The homologous repair which is carried out by the NCLDV is a process of homologous recombination which is carried out by said virus to restore complementarity of its Inverted Terminal Repeats (ITRs). This complementarity ensures the ability of the NCLDV to replicate/propagate in host cells and/or improves stability of the NCLDV. The process of homologous repair can also be designated as self-correction, copy-correction or homology-directed repair (HDR).

In one embodiment, the cell line transfected in step (i) is produced by infecting the cell line with a nucleocytoplasmic large DNA virus (NCLDV). This step is specifically carried out before step (i).

In one another embodiment, the regions which flank the (heterologous) nucleotide sequence of interest allow homologous recombination within the center of the Inverted Terminal Repeats (ITRs) of the NCLDV. The regions which flank the (heterologous) nucleotide sequence of interest particularly allow stable homologous recombination within the center of the Inverted Terminal Repeats (ITRs) of the NCLDV.

The cell line may be an adherent cell line or a non-adherent cell line, e.g. a cell line which grows in suspension culture, specifically under serum-free conditions.

Preferably, the cell line is selected from the group consisting of an avian cell line, preferably a chicken retina cell line AGE1.CR.PIX, a DF-1 chicken fibroblast cell line, or a duck embryonic stem cell line EB66, a mammalian cell line, preferably a baby hamster kidney (BHK) cell line or a human embryonal kidney 293 (HEK293) cell line, an arthropod cell line, preferably a Sf9 cell line, and a piscine cell line, preferably a SAF-1 cell line.

Thus, in one preferred embodiment, the present invention relates to a method for producing a nucleocytoplasmic large DNA virus (NCLDV) containing a polynucleotide comprising an identical (heterologous) nucleotide sequence of interest within each (of the two) Inverted Terminal Repeat(s) (ITR(s)) comprising the steps of:
(i) transfecting an avian or a mammalian cell line infected with a nucleocytoplasmic large DNA virus (NCLDV) with a (heterologous) nucleotide sequence of interest, wherein the (heterologous) nucleotide sequence of interest is flanked by regions capable of homologous recombination within the Inverted Terminal Repeats (ITRs) of the NCLDV, thereby obtaining a NCLDV containing a polynucleotide comprising a (heterologous) nucleotide sequence of interest within one of the ITRs, and
(ii) culturing the NCLDV virus obtained in (i) under conditions allowing homologous repair between the Inverted Terminal Repeats (ITRs), thereby obtaining a NCLDV containing a polynucleotide comprising an identical (heterologous) nucleotide sequence of interest within each (of the two) Inverted Terminal Repeat(s) (ITR(s)).

Preferably, the NCLDV is a virus of the Poxviridae, Asfarviridae, or Iridoviridae family.

More preferably,
the virus of the Poxviridae family is selected from the group consisting of a vaccinia virus, an avipoxvirus, and a parapoxvirus,
the virus of the Asfarviridae family is an asfivirus, or
the virus of the Iridoviridae family is a lymphocystis disease virus (LCDV) or a Chloriridovirus.

Even more preferably,
the vaccinia virus is an attenuated vaccinia virus selected from the group consisting of a Modified Vaccinia Ankara (MVA) virus, an MVA related virus, a vaccinia virus Dairen-I, and
a vaccinia virus strain NYVAC,
the avipoxvirus is selected from the group consisting of a canarypox virus and a fowlpox virus, or
the parapoxvirus is an ORF virus.

Thus, in one more preferred embodiment, the present invention relates to a method for producing an MVA virus containing a polynucleotide comprising an identical (heterologous) nucleotide sequence of interest within each (of the two) Inverted Terminal Repeat(s) (ITR(s)) comprising the steps of:
(i) transfecting an avian cell line infected with an MVA virus with a (heterologous) nucleotide sequence of interest, wherein the (heterologous) nucleotide sequence of interest is flanked by regions capable of homologous recombination within the Inverted Terminal Repeats (ITRs) of the MVA virus, thereby obtaining an MVA virus containing a polynucleotide comprising a (heterologous) nucleotide sequence of interest within one of the ITRs, and
(ii) culturing the MVA virus obtained in (i) under conditions allowing homologous repair between the Inverted Terminal Repeats (ITRs), thereby obtaining an MVA virus containing a polynucleotide comprising an identical (heterologous) nucleotide sequence of interest within each (of the two) Inverted Terminal Repeat(s) (ITR(s)).

Specifically, the MVA virus comprises a left Inverted Terminal Repeat (ITR) and a right Inverted Terminal Repeat (ITR). Thus, for example, transfection of an avian cell line infected with an MVA virus with a (heterologous) nucleotide sequence of interest, wherein the (heterologous) nucleotide sequence of interest is flanked by regions capable of homologous recombination within the Inverted Terminal Repeats (ITRs) of the MVA virus, results in/leads to an MVA virus containing a polynucleotide comprising a (heterologous) nucleotide sequence of interest in the right Inverted Terminal Repeat (ITR). Subsequent culture of the MVA virus under conditions allowing homologous repair between the two ITRs further results in/leads to an MVA virus containing a polynucleotide comprising a (heterologous) nucleotide sequence of interest also in the left Inverted Terminal Repeat (ITR).

Alternatively, transfection of an avian cell line infected with an MVA virus with a (heterologous) nucleotide sequence of interest, wherein the (heterologous) nucleotide sequence of interest is flanked by regions capable of homologous recombination with the Inverted Terminal Repeats (ITRs) of the MVA virus, results in/leads to an MVA virus containing a polynucleotide comprising a (heterologous) nucleotide sequence of interest in the left Inverted Terminal Repeat (ITR). Subsequent culture of the MVA virus under conditions allowing homologous repair between the two ITRs further results in/leads to an MVA virus containing a polynucleotide comprising a (heterologous) nucleotide sequence of interest also in the right Inverted Terminal Repeat (ITR).

The left ITR of the MVA virus as mentioned above ranges, for example, from nucleotide position 166 to 9809 or from a nucleotide position corresponding thereto of the polynucleotide according to Genbank Accession number U94848 (version U94848.1 and GI: 2772662) and/or the right ITR of the MVA virus as mentioned above ranges, for example, from nucleotide position 168280 to 177923 or from a nucleotide position corresponding thereto of the polynucleotide according to Genbank Accession number U94848 (version U94848.1 and GI: 2772662).

Particularly, the regions which flank the (heterologous) nucleotide sequence of interest allow homologous recombination within the center of the Inverted Terminal Repeats (ITRs) of the MVA virus. More particularly, the regions which flank the (heterologous) nucleotide sequence of interest allow stable homologous recombination within the center of the Inverted Terminal Repeats (ITRs) of the MVA virus. The center of the ITRs can be characterized by a distance corresponding to half the width of the ITR+/−10% of the width, or +/−20% of the width, or +/−30% of the width. For example, the center of the ITRs is 7.5+/−1.5 kbp from the sealed genomic termini for a wildtype MVA.

In the final MVA virus, the identical (heterologous) nucleotide sequences of interest are preferably comprised at mirror inverted identical positions within the (center of the) ITRs. More preferably, the MVA virus produced with the method according to the second aspect is an MVA virus according to the first aspect.

In one another more preferred embodiment, the present invention relates to a method for producing an MVA related virus containing a polynucleotide comprising an identical (heterologous) nucleotide sequence of interest within each (of the two) Inverted Terminal Repeat(s) (ITR(s)) comprising the steps of:
(i) transfecting an avian cell line infected with an MVA related virus with a (heterologous) nucleotide sequence of interest, wherein the (heterologous) nucleotide sequence of interest is flanked by regions capable of homologous recombination within the Inverted Terminal Repeats (ITRs) of the MVA related virus, thereby obtaining an MVA related virus containing a polynucleotide comprising a (heterologous) nucleotide sequence of interest within one of the ITRs, and
(ii) culturing the MVA related virus obtained in (i) under conditions allowing homologous repair between the Inverted Terminal Repeats (ITRs), thereby obtaining an MVA related virus containing a polynucleotide comprising an identical (heterologous) nucleotide sequence of interest within each (of the two) Inverted Terminal Repeat(s) (ITR(s)).

Specifically, the MVA related virus contains a polynucleotide comprising two copies of a nucleotide sequence comprising deletion site IV and the right ITR. Accordingly, the regions which flank the (heterologous) nucleotide sequence of interest particularly allow homologous recombination within deletion site IV of the Inverted Terminal Repeats (ITRs) of the MVA related virus. More particularly, the regions which flank the (heterologous) nucleotide sequence of interest allow stable homologous recombination within deletion site IV of the Inverted Terminal Repeats (ITRs) of the MVA related virus. Thus, for example, transfection of an avian cell line infected with an MVA related virus with a (heterologous) nucleotide sequence of interest, wherein the (heterologous) nucleotide sequence of interest is flanked by regions capable of homologous recombination within the Inverted Terminal Repeats (ITRs) of the MVA related virus, results in/leads to an MVA related virus containing a polynucleotide comprising a (heterologous) nucleotide sequence of interest in one copy of deletion site IV. Subsequent culture of the MVA virus under conditions allowing homologous repair between the two ITRs further results in/leads to an MVA virus containing a polynucleotide comprising a (heterologous) nucleotide sequence of interest also in the other copy of deletion site IV. More particularly, the (two) deletion sites IV are positioned in the center of the ITRs. The center of the ITRs can be characterized by a distance corresponding to half the width of the ITR+/−10% of the width, or +/−20% of the width, or +/−30% of the width. For example, the center of the ITRs is 13.5 kbp+/−2.7 kbp for the MVA related virus (MVA-CR19). For the MVA related virus (MVA-CR19), the distance of DS IV to the proximal terminus is 11 kbp, and the distance to the end of the repeat and start of the genome core is 16 kbp. The one ITR ranges, for example, from nucleotide position 1 to 26920 or from a nucleotide position corresponding thereto of the polynucleotide according to Genbank Accession number KY633487 (version KY633487.1) and/or the other ITR ranges, for example, from nucleotide position 163630 to 190549 or from a nucleotide position corresponding thereto of the polynucleotide according to Genbank Accession number KY633487 (version KY633487.1).

In the above embodiments, the deletion site IV is preferably comprised in the center of the ITRs.

In the final MVA virus produced with the method according to the second aspect, the MVA related virus preferably contains a polynucleotide comprising the identical (heterologous) nucleotide sequences of interest in each deletion site IV. In other words, the MVA related virus preferably contains two copies of a nucleotide sequence comprising deletion site IV and the right ITR and wherein one of the identical (heterologous) nucleotide sequences of interest is comprised in one copy of deletion site IV and the other one of the identical (heterologous) nucleotide sequences of interest is comprised in the other copy of deletion site IV. More preferably, the MVA related virus produced with the method according to the second aspect is an MVA related virus according to the first aspect.

The nucleotide sequence of interest (which is flanked by regions capable of homologous recombination) which is transfected in step (i) of the method according to the second aspect may be part of/comprised in a plasmid/vector such as shuttle vector.

Preferably, the nucleotide sequence of interest is under control of/operably linked to a promoter. More preferably, the promoter is a NCLDV specific promoter. In case of a poxvirus, the NCLDV specific promoter may be a poxviral promoter. Even more preferably, the nucleotide sequences of interest are capable of being expressed from the NCLDV.

Specifically, the nucleotide sequences of interest are selected from the group consisting of a sequence coding for an antigen, particularly an epitope of an antigen, a diagnostic compound, and a therapeutic compound. More specifically, the therapeutic compound is a virotherapeutic compound, a vaccine, or an oncolytic compound.

The NCLDV produced with the method according to the second aspect is preferably a NCLDV according to the first aspect.

Culturing under conditions allowing homologous repair is performed as follows: A permissive cell line is infected with the NCLDV containing the nucleotide sequence of interest in one ITR. In a permissive cell line, genomic replication occurs that allows the one copy of the nucleotide sequence of interest in one ITR to contact the other ITR at the corresponding identical position, particularly mirror inverted identical position. Active genomic replication in the permissive infected cell line leads to homologous repair within 1 to 10 passages. Each passage is defined by infection with the NCLDV containing the nucleotide sequence of interest in at least one ITR, isolation of the virus by cell lysis, and reinfection.

The culturing time may range from between 1 day and 24 days, e.g. 1, 2, 3, 4, 5, 6, 7, 8, 9, 10, 11, 12, 13, 14, 15, 16, 17, 18, 19, 20, 21, 22, 23, or 24 day(s).

The NCLDV culture may be an agitated culture or a culture in a bioreactor. Bioreactors are generally categorized similarly to chemical reactors according to their mixing characteristics. Said bioreactor may be a (well mixed) stirred tank reactor or a plug flow (tubular) reactor. In an ideal well-mixed bioreactor, the mixing is assumed to be intense enough that the fluid (cells and culture medium) is homogenous through the reactor. The bioreactor may be a fed-batch, batch, or continuous bioreactor or the culturing process may be a batch, fed-batch or continuous culturing process.

The method may further comprise the step of isolating/harvesting the produced/generated NCLDV from said cell line.

Various isolation/harvesting procedures are known in the art for NCLDV which escape their host cells after their production. For example, the isolation/harvesting is achieved by separating the NCLDV from the cells via centrifugation, sedimentation and/or filtration, e.g. via centrifugation and filtration, via sedimentation and filtration, via sedimentation and centrifugation, or via centrifugation, sedimentation, and filtration. Depending on the NCLDV virus to be harvested, the parameters for centrifugation, sedimentation or filtration may vary. The person skilled in the art is able to easily adapt the appropriate separation parameters, e.g. the acceleration-force/G-force and/or time using centrifugation for separation, filter size using filtration for separation, and/or sedimentation time using sedimentation for separation, in order to harvest the NCLDV produced by said cells.

In a third aspect, the present invention relates to a nucleocytoplasmic large DNA virus (NCLDV) obtainable by the method according to the second aspect.

In a fourth aspect, the present invention relates to a method for propagating a nucleocytoplasmic large DNA virus (NCLDV) according to the first or third aspect comprising the step of:
culturing the NCLDV according to the first or third aspect in a cell line.

The cell line may be an adherent cell line or a non-adherent cell line, e.g. a cell line which grows in suspension culture, specifically under serum-free conditions.

Preferably, the cell line is selected from the group consisting of an avian cell line, preferably a chicken retina cell line AGE1.CR.PIX, a DF-1 chicken fibroblast cell line, or a duck embryonic stem cell line EB66, a mammalian cell line, preferably a baby hamster kidney (BHK) cell line or a human embryonal kidney 293 (HEK293) cell line, an arthropod cell line, preferably a Sf9 cell line, and a piscine cell line, preferably a SAF-1 cell line.

Culturing may be performed according to standard procedures readily available to the skilled person. In this respect, is should be noted that culture/incubation times are important for the production of the NCLDV in high yields. In addition, since the integral of viable cell density (IVCD) is one of the factors that impact the overall volumetric productivity in cell culture, it is desirable to keep cell viability at a high level over the duration of upstream processing. Further, nutrient depletion, extended exposure to impeller shear forces and other factors that occur during culturing may constitute apoptotic stimuli which serve to decrease cell viability and which should, thus, be avoided.

The culturing time may range from between 1 day and 24 days, e.g. 1, 2, 3, 4, 5, 6, 7, 8, 9, 10, 11, 12, 13, 14, 15, 16, 17, 18, 19, 20, 21, 22, 23, or 24 day(s).

The NCLDV culture may be an agitated culture or a culture in a bioreactor. Bioreactors are generally categorized similarly to chemical reactors according to their mixing characteristics. Said bioreactor may be a (well mixed) stirred tank reactor or a plug flow (tubular) reactor. In an ideal well-mixed bioreactor, the mixing is assumed to be intense enough that the fluid (cells and culture medium) is homogenous through the reactor. The bioreactor may be a fed-batch, batch, or continuous bioreactor or the culturing process may be a batch, fed-batch or continuous culturing process.

The method may further comprise the step of isolating/harvesting the propagated NCLDV from said cell line.

Various isolation/harvesting procedures are known in the art for NCLDV which escape their host cells after their production. For example, the isolation/harvesting is achieved by separating the NCLDV from the cells via centrifugation, sedimentation and/or filtration, e.g. via centrifugation and filtration, via sedimentation and filtration, via sedimentation and centrifugation, or via centrifugation, sedimentation, and filtration. Depending on the NCLDV virus to be harvested, the parameters for centrifugation, sedimentation or filtration may vary. The person skilled in the art is able to easily adapt the appropriate separation parameters, e.g. the acceleration-force/G-force and/or time using centrifugation for separation, filter size using filtration for separation, and/or sedimentation time using sedimentation for separation, in order to harvest the NCLDV produced by said cells.

In a fifth aspect, the present invention relates to a pharmaceutical composition comprising a nucleocytoplasmic large DNA virus (NCLDV) according to the first or third aspect.

The pharmaceutical composition comprising an NCLDV according to the first or third aspect may comprise one or more excipient(s), diluent(s), and/or carrier(s), all of which are preferably pharmaceutically acceptable.

The pharmaceutical composition can be administered systemically, e.g. parenterally. For example, the pharmaceutical composition can be in a form suitable for oral administration, nasal administration, or administration by inhalation. The pharmaceutical composition can also be administered intravascular, intravenous, intramuscular, intrathecal, subcutaneous, or intraperitoneal.

The pharmaceutical composition can be administered in a single dose or in more than one dose. It is preferred that the pharmaceutical composition is to be administered in a therapeutically effective amount.

In a sixth aspect, the present invention relates to a nucleocytoplasmic large DNA virus (NCLDV) according to the first or third aspect, or a pharmaceutical composition according to the fifth aspect for use in medicine.

For example, the NCLDV according to the first or third aspect or the pharmaceutical composition according to the fifth aspect is used in therapy such as oncotherapy, vaccination therapy, or gene therapy.

In a seventh aspect, the present invention relates to the present invention relates to a nucleocytoplasmic large DNA virus (NCLDV) according to the first or third aspect, or a pharmaceutical composition according to the fifth aspect for use in therapy.

Preferably, the therapy is gene therapy, vaccination therapy, or oncotherapy.

The seventh aspect of the present invention can alternatively be worded as follows: A method for treating a subject comprising the step of: administering a NCLDV according to the first or third aspect or a pharmaceutical composition according to the fifth aspect to a subject (in need thereof), thereby treating the subject.

The seventh aspect of the present invention can further alternatively be worded as follows: Use of a NCLDV according to the first or third aspect or a pharmaceutical composition according to the fifth aspect for the manufacture of a medicament for treating a subject.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: Genomic organisation of wildtype MVA and strain CR19. The terminal repeats have expanded in size in MVA-CR19 by a recombination event that has caused loss of deletion site (DS) I at the left side and duplication of DS IV at both sides of the genome.

FIG. 2: Possible genotypes and phenotypes for recombination of different markers into deletion site (DS IV) of wildtype MVA and MVA-CR19. The two ITRs are drawn as arrow boxes. DS IV is localized outside of the right ITR of wildtype (shown as downpointing arrow) and within both ITRs of MVA-CR19. DS I is localized outside of the left ITR of wildtype and not present in MVA-CR19. Open boxes are for parental ITRs. The letters in filled arrow boxes of MVA-CR19 stand for insertion of EGFP and mCherry (Red). The bp numbers refer to the sizes of diagnostic PCR products obtained with the shown configuration using primers SEQ ID 9 and 10 (for DS I) and SEQ ID 5 and 6 (for DS IV).

FIG. 3: Plaques that appeared to be yellow but are shown to be formed by different co-infecting viruses at higher magnification. The arrows in the EGFP and mCherry panels point to plaques that overlap only partially in the merged panel and thus are not of a common origin.

FIG. 4: Relative composition of plaque populations isolated after simultaneous recombination of WT virus with shuttle plasmids for EGFP and mCherry reporters. The numbers in the columns give absolute plaque numbers. Shown here is the development of one green (g1), red (r1) and six independently picked yellow (y1-y6) plaques. Red and green plaques could be purified within 5 passages (P1-P5), yellow plaques always dispersed into a mixture of red and green plaques in addition to yellow plaques.

FIG. 5: Relative composition of plaque populations isolated after simultaneous recombination of MVA-CR19 virus with shuttle plasmids for EGFP and mCherry reporters. The numbers in the columns give absolute plaque numbers. As shown in parallel with wildtype MVA, development of one green (g1), red (r1) and six independently picked yellow (y1-y6) plaques is quantified. Red and green plaques could be purified, yellow plaques always dispersed into a mixture of red and green plaques in addition to yellow plaques.

FIG. 6: MVA-CR19 tolerates inserts at the duplicated DS IV in the viral telomers. The expected amplicons are 291 bp for wildtype (empty) DS I, 1099 bp for 4 PT-RED in DS IV, 1231 bp for 45T-GFP in DS IV, 220 bp for wildtype (empty) DS IV, and 702 bp for wildtype DS VI (see also FIG. 2). Non-template controls were without signals and are on a separate gel (not shown here). Primers are SEQ ID 9 and 10 (for DS I), SEQ ID 5 and 6 (for DS IV) and SEQ ID 7 and 8 (for DS VI).

FIG. 7: WT RED x GFP Chart. Phenotype distribution of plaques after recombination of WT (A) and CR19 viruses (B) with mCherry in DS IV with a shuttle plasmid that codes for EGFP. Yellow plaques were picked into pools (py) at passages 1 and 2. The plaques were not pooled at passages 3-5 (individually named y1-y5). For WT, all yellow pools and yellow clones separated into green, red and yellow components independent of passage level. For CR19, a clone was identified in P3 (y1) that had no apparent red components and very high frequency of yellow. That clone was plaque-picked for further 2 passages (P4 and P5, P4* is an independent passage of the same clone).

FIG. 8: Insertion at DS IV has increased in size for yellow plaque. Recombination may have occured by a chance integration of different inserts at the distal termini. Intermediate bands are visible and marked with asterisks. Source for the plaques shown in the right panel are plaques from P4* in FIG. 7.

FIG. 9: The large insertion is stably maintained for at least 5 passages. Insertion at DS IV has increased in size for yellow plaque.

FIG. 10: Separation of GFP and mCherry signals in a mixed population but not in the clone containing the single large insert. Both, comets and methylcellulose-confined plaques show frequent separation into monofluorescent colors in the population with the mixed genotype (some highlighted with arrows in the panels for EGFP and mCherry). The yellow clone shows only congruent fluorescence for both colors.

FIG. 11: Genotype of P1 plaques that were picked for a red phenotype in a replacement of an EGFP-1 expression cassette in MVA-CR19 against a mCherry expression cassette in a shuttle plasmid for DS IV.

FIG. 12: Quantification of plaques that were obtained in a replacement of an EGFP-1 expression cassette in DS IV of MVA-CR19 against an mCherry expression cassette. (a) Plaques with a predominantly monofluorescent red phenotype were obtained after 2 passages. A pure yellow phenotype was not recovered and high frequency of red or green plaques were observed for all 14 of isolated yellow P3 plaques. (b) A representative section of the wells in the 6-well-plate, here for plaque P3 y06. A large number of plaques can be seen in the EGFP panel that do not have a corresponding signal in the mCherry panel, and vice versa. These plaques do not appear as a yellow signal in the merged panels

FIG. 13: Confirmation that recombination towards large dual-expressing insert is infrequent. Plaque r01 is the mixed-fluorescence plaque P2 r1 in FIG. 12, y10* is the incomplete yellow plaque from passage 5, see FIG. 9. Plaque y01 was also compared to a pure yellow plaque in a comet assay, see FIG. 10. RED and GFP are the positive controls as described above. Expected amplicons are 1099 bp for DS IV containing mCherry, 1231 bp for DS IV containing GFP, approx. 2000 bp for a combination of both reporter genes, and 220 bp for an empty (wildtype) DS IV (with primers SEQ ID 5 and 6).

FIG. 14: Expression cassettes in DS IV of strain CR19 double the gene dosis and thus expression strength at superior genetic stability compared to wildtype. The tested expression cassettes are 45dxT-Dual (containing the P11 promoter for mCherry and mH5 promoter for EGFP) and 4 PT-RED. The signals for mCherry in the dual expression cassette are lower compared to the single expression cassette because of promoter interference.

### EXAMPLES

The examples given below are for illustrative purposes only and do not limit the invention described above in any way.

### Example 1

### Design of a Shuttle Vector for Deletion Site (DS) IV

The shuttle vector pSh III ELP11 Dual for deletion site (DS) III has been described previously (Jordan et al. 2019). The shuttle vector for deletion site (DS) IV described here is a derivative therefrom. The flanks were obtained by PCR on viral genomic DNA. Insertion into the backbone of the DS III shuttle vector was done with restriction sites PciI, NheI, NotI or DraIII that were contained in the primers (all restriction enzymes were from New England Biolabs). Sequence of all PCR-amplified regions was confirmed by Sanger sequencing of the final plasmids. The primers used for cloning of the left flank (820 bp) via PciI and NheI for the shuttle plasmid for DS IV are SEQ ID 1 and SEQ ID 2, and for cloning of the right flank (888 bp) via NotI and DraIII are SEQ ID 3 and SEQ ID 4. PCR was performed in a final volume of 25 μL with 0.15 μL Taq polymerase (Qiagen, Germany), 200 nmol/L each primer, and 125 μmol/L each nucleotide. Thermocycling was initiated with 94 °C for 80 s, followed by 35 cycles of 94 °C for 20 s, 55 °C for 20 s and 72 °C for 90 s, and terminated with 72 °C for 5 min.

Screening for the insertion in DS IV was done with primers SEQ ID 5 and SEQ ID 6. The sizes are 220 bp in wildtype MVA, 2016 bp for the dual expression construct, 1231 bp for the construct with GFP and 1099 bp with mCherry.

Screening for the insertion in DS VI was done with primers SEQ ID 7 and SEQ ID 8. Screening for presence of DS I was done with primers SEQ ID 9 and SEQ ID 10.

### Example 2

Simultaneous Dual Insertion: Occupancy of DS IV does not Interfere with Viability of MVA-CR19

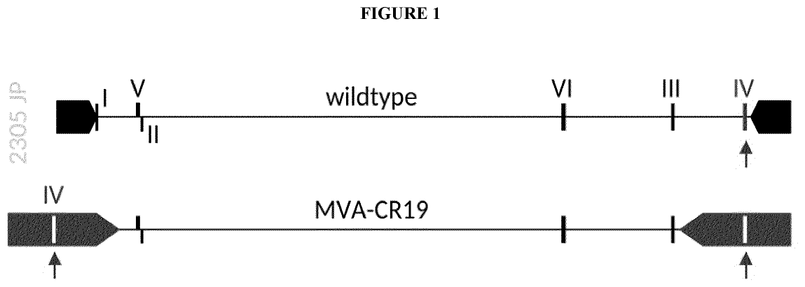

A prominent feature of MVA-CR19, a MVA related virus, is the large terminal duplication that also extends to DS IV (Jordan et al. 2019). FIG. 1 depict the genome of wildtype MVA and MVA-CR19 used in this and following studies. DS IV shuttle plasmids were created for recombination of expression cassettes of either mCherry or EGFP. The terminal repeats function as the origin of replication for genome amplification (Senkevich et al. 2015) and large changes to these structures can render a virus unstable (Dobson and Tscharke 2015). Up to date, it was not known whether recombination into the duplicated (repeat) regions in the viral genome is possible at all. It is novel to test whether inserts are tolerated in the viral terminal repeats, and whether different inserts are possible.

Three complementary approaches were investigated to obtain MVA-CR19 with different inserts in the two terminal DS IV elements: simultaneous recombination of parental CR19 with shuttle plasmids for GFP and mCherry (p45T-GFP and p4PT-RED), and consecutive recombinations of GFP into a mCherry-expressing virus (MVA-CR19.45T-GFP x p4PT-RED) and vice versa (MVA-CR19.4 PT-RED x p45T-GFP). All combined, more than 80 plaques were isolated and tested by PCR or expression of fluorescent markers to obtain a total of more than 70000 data points. No red and green (“yellow”) viruses could be recovered with a single exception. The single exception was a virus with a recombination that expanded in size such that that both reporter genes were contained in both termini as identical copies of the final expression cassette.

In detail, for the simultaneous recombination 1×10\hat{ }6 adherent CR.pIX cells (Jordan et al. 2009) in DMEM:F12 (Gibco) with 5% FCS (Gibco) in a well of a six-well plate were infected with wildtype (WT) MVA or MVA-CR19 with an MOI of 0.01. The two wells were transfected with 1 μg each of the GFP and mCherry shuttle plasmids 90 min after infection. Transfection was done with Effectene (Qiagen) according to the manufacturer's instructions. Medium was replaced 5 h post transfection. Appearance of the cultures 3 days post infection/transfection gave clear indications of the expected virus phenotypes, spontaneous syncytia in the well with wildtype and scattered signals in the well with parental CR19. Expression of EGFP or mCherry is under control of poxviral promoters and therefore possible only if an infected cell is transfected. Co-transfection efficiency appeared to be high in a digital overlay of green and red fluorescence images.

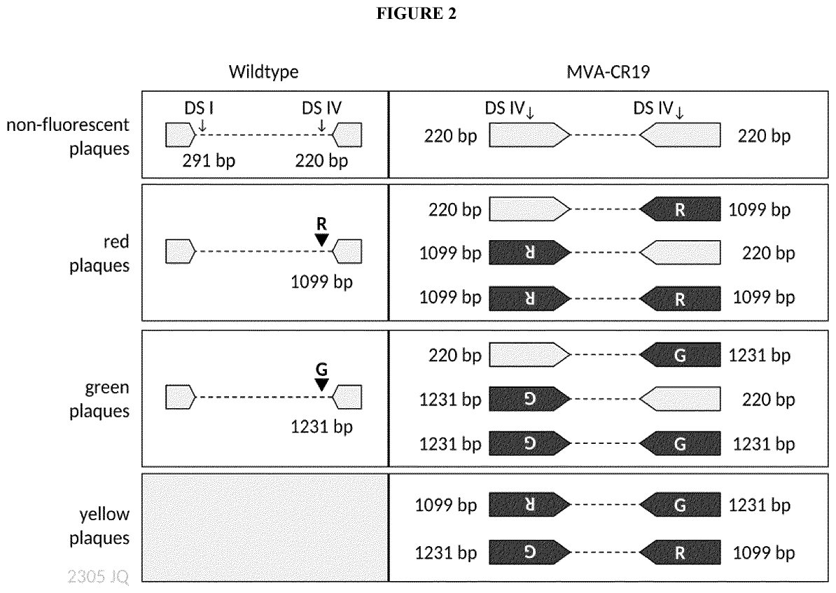

A cryogenic lysate was obtained, and this lysate was furthermore sonicated with a VialTweeter (Hielscher) set to 20 s of 100% cycle and 90% amplitude. A dilution series of the two lysates was prepared and volumes corresponding to 2 μL, 0.2 μL, 0.02 μL of the 500 μL volumes were used to infect fresh monolayers in 6-well plates (resulting in passage 1 (P1) plaques). Methylcellulose (Sigma) to 1% was applied after 5 h to prevent diffusion of progeny virus and to facilitate subsequent plaque picking. The P1 plates were scanned 3 days post infection. The possible genotypes and phenotypes for recombination of two different markers into DS IV of wildtype MVA and MVA-CR19 is shown in FIG. 2.

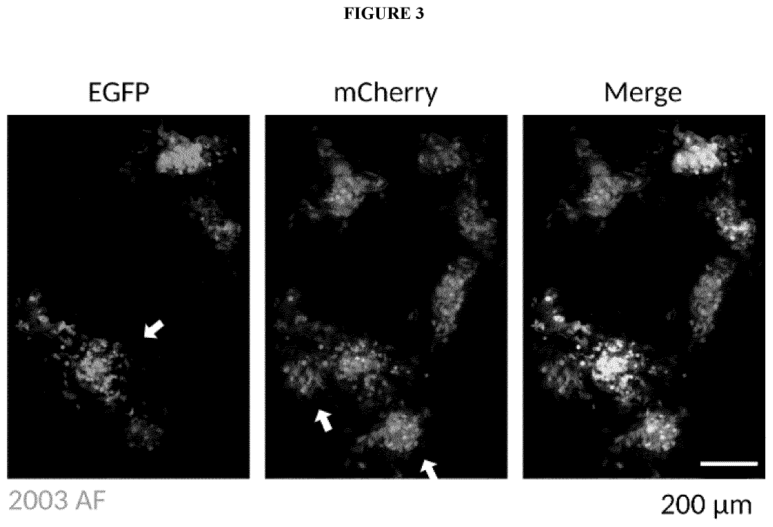

As expected, most plaques were caused by infection with parental virus and, thus, were without reporter-gene expression. Plaques that expressed reporter often were mono-fluorescent red or green. Yellow plaques were found in recombination experiments with both wildtype (that is equipped with only one DS IV) and CR19. A majority of the yellow plaques were consistent with being obtained by co-infection of viruses that express either GFP or mCherry because some of the methylcellulose-confined regions did not express both markers when viewed at greater magnification (FIG. 3).

From both experimental threads red, green and yellow plaques were picked by aspiration of approx. 20 μL volumes with a 100 μL pipette tip, transferred to 500 μL of medium, sonified, and used to infect fresh monolayers in 6-well plates at 5-fold dilution (100 μL/500 μL) (P2). Methylcellulose was added 5 h post infection. Two days post infection, again several monofluorescent and yellow plaques each were picked for wildtype and CR19 and used for a next serial passaging step at 10-fold dilution (P3). Similarily, monofluorescent and yellow plaques were picked for a fourth passaging step. The number of plaques in this and subsequent experiments were quantified by automated counting using the experimental NyOne Fluorescent Plaque Morphology (2F) (v. 0.9) module and a NyOne Scientific (Synentec) cell imager. The software module returns number and area (in mm\hat{ }2) of fluorescent plaques for each channel (here, green and red) and the total fluorescent area. Yellow (mixed channels) plaques were calculated by first dividing the fluorescent area for each channel by the number of plaques for that channel to obtain the mean area (size) of a plaque for that particular channel. Next, the combined area of fluorescence for both channels was divided by the mean area of plaques of one channel. This returns the number of plaques of a color (green or red) that contribute to the combined (yellow) plaques.

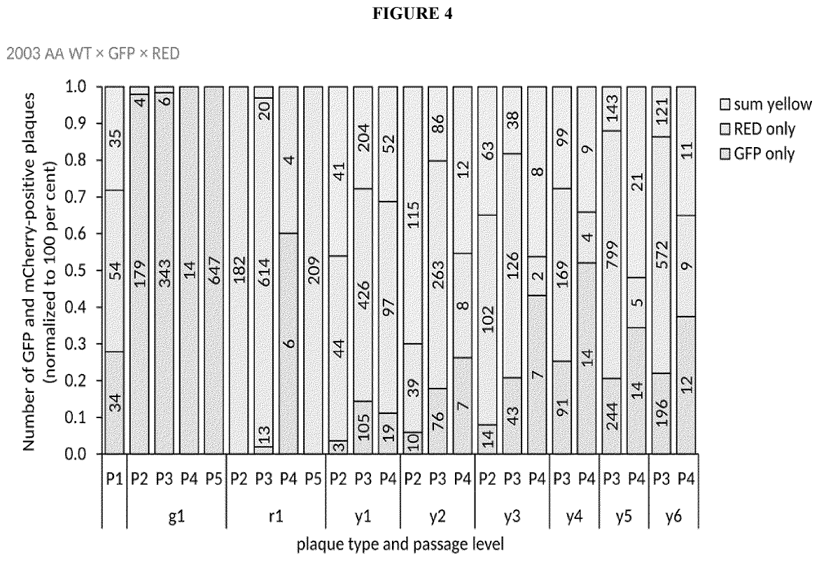

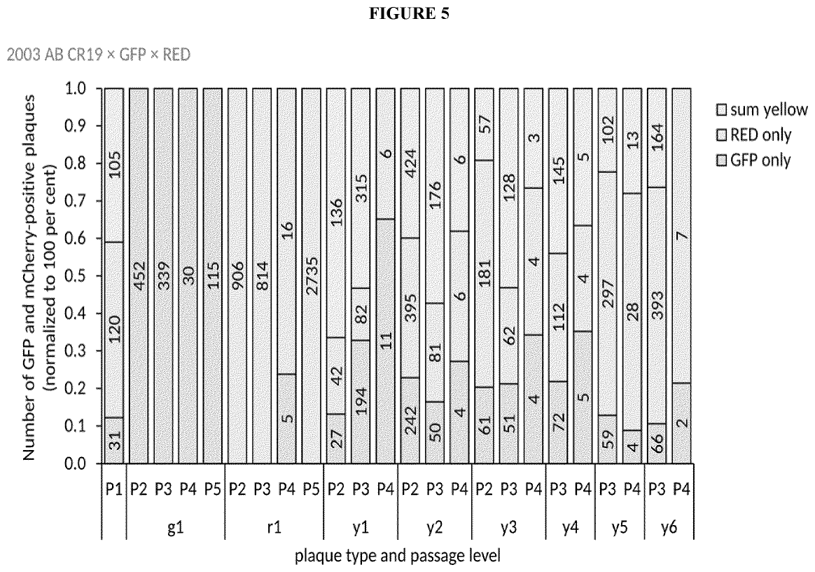

As shown in FIG. 4 and FIG. 5, pure monofluorescent plaques, either red or green, were easily obtained within 2-5 passages for both wildtype and strain CR19 viruses. However, all yellow plaques separated into green and red plaques (in addition to yellow plaques) in subsequent passages.

Recombination of viral genome with ectopic DNA requires active genome replication. At the onset of this study it was expected that recombinant (foreign) inserts in the terminal repeats are not tolerated at all (because of indications in the literature that these may destabilise the replication signals) or mechanistically not possible (because the terminal repeats may be engaged in multi-strand helices and occupied by the replication machinery). One important conclusion is that red or green mono-fluorescent viruses were obtained in this experiment out of an initially mixed recombination. This demonstrates that recombination of ectopic sequences into DS IV is possible in MVA-CR19 and, thus, into a region that is duplicated at both termini of the genome and important for replication of the virus.

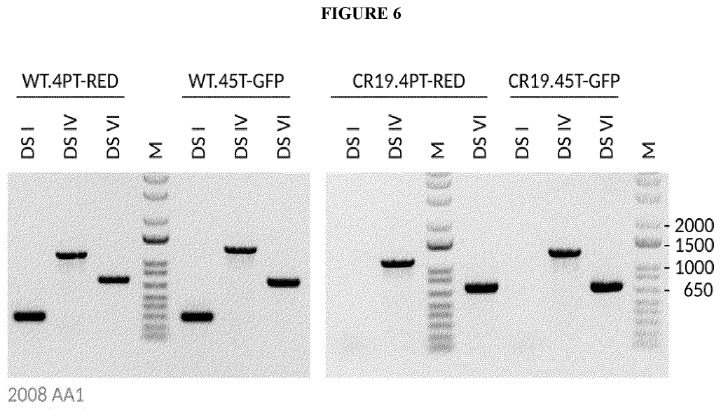

The isolated monoflurescent viruses of passage 4 were named according to receiving virus and shuttle plasmid MVA-WT.4 PT-RED, MVA-WT.45T-GFP, MVA-CR19.4 PT-RED and MVA-CR19.45T-GFP. They were used to infect fresh monolayers of CR.pIX cells in 6-well plates. The progeny viruses were tested 48 h post infection by analysis of fluorescence distribution (FIG. 4 and FIG. 5, results for P5) and by PCR analysis (FIG. 6). As summarised in FIG. 1, presence of DS I allows a differentiation of WT and CR19. Clear signals for DS I were visible with the genomic DNA of WT viruses and no signal for DS I was visible with genomic DNA of CR19 as template. PCR with primers against DS VI served as internal control and gave the expected amplicon. Consistent with the monofluorescence phenotype, an occupancy with either mCherry or EGFP in DS IV was confirmed. Importantly, no indications of an empty DS IV (either because of contamination with parental virus or due to later deletions) were detected. A full lysate was generated of each virus to continue a study based on consecutive insertion of genes of interest into the ITR.

### Example 3

Consecutive Insertion of EGFP into mCherry-Viruses Confirms Homology-Directed Repair

Since no CR19-recombinants could be isolated that express both GFP and mCherry, as an alternative approach consecutive recombination of the complementary reporter into an already singly recombinant virus was pursued. The receiving viruses were obtained by infection of 6-well cultures with 250 μL (out of 500 μL) each of P4 red and green isolates (now P5).

The first series initiated with recombination of p45T-GFP into wildtype and MVA-CR19 viruses that already contain mCherry in DS IV. Because DS IV is duplicated in MVA-CR19, three outcomes can be expected: GFP replaces DSRed1 at both telomers (resulting in a green plaque, G-G in an abbreviated notation with G for GFP at bot termini), mCherry is fully maintained (red plaque, R-R with R for mCherry), or insertion of GFP at one telomer while mCherry is maintained at the other telomer (resulting in a plaque with mixed colors, here termed yellow, with R-G or G-R as potential genotype). If the two telomers communicate then conversion can be expected from yellow to either single color (red or green, not both; from R-G/G-R to R-R or G-G).

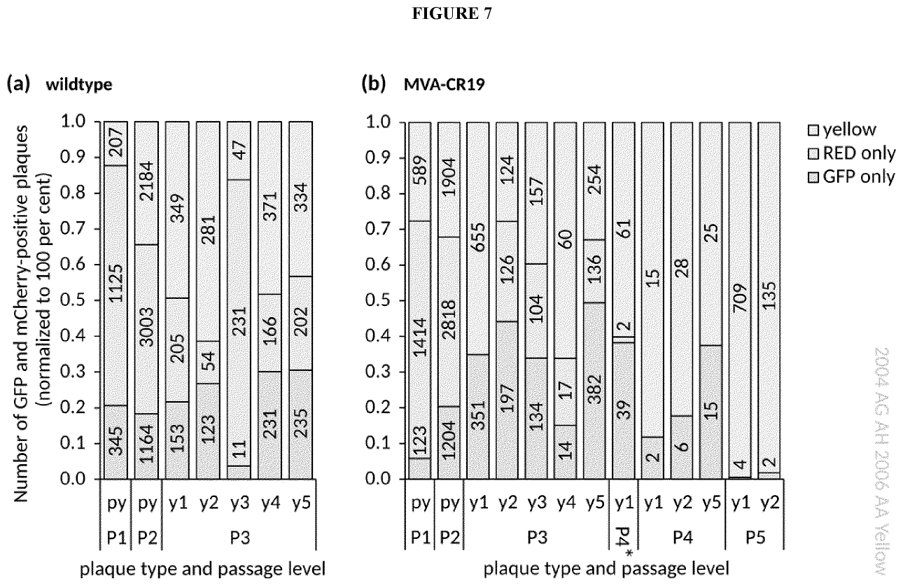

The mCherry-recombinant viruses (MVA-A3.4 PT-RED and MVA-CR19.4 PT-RED) were recombined with p45T-GFP shuttle plasmid each in a well of a 6-well plate as described previously. Transfection efficiency again was high and the lysate was transferred to a fresh 6-well plate in volumes from 2 μL down to 1 nL. Virus diffusion was inhibited by methylcellulose as described above and each passage was quantified by the automated NyOne plaque counting (FIG. 7). After 2 days, 7 (WT) and 38 (CR19) yellow P1 plaques were picked out of a background of predominantely red plaques (contaminating parental virus) and green plaques. The plaques were pooled into 1 mL of medium each for the next passage.

In addition, 9 mono-fluorescent green CR19 plaques were picked into 20 μL medium each and analysed by PCR with primers SEQ ID 5 and SEQ ID 6.

Fresh monolayers of CR.pIX cells in 6-well plates were infected with the pooled 7 green WT plaques with volumes from 200 μL down to 1:200, and with the pool of 38 yellow plaques with volumes of 50 μL down to 1:250. Methylcellulose was applied after 5 h. No parental (non-fluorescent) WT or CR19 viruses were detected. Results of the automated counting using the NyOne Scientific cell imager returned a total of 6351 plaques for WT and 5927 for CR19 in all dilutions combined. The majority of the obtained plaques are monofluorescent red (parental virus, 82 and 86% for WT and CR19). Although yellow plaques were initially picked, only 54% of the WT GFP and 57% of the CR19 GFP plaques were yellow (14 and 18% of the red plaques), and no obvious differences were measured between WT and CR19 (with one vs. two deletion sites IV).

Five yellow plaques each of wildtype and strain CR19 were picked and 25 μL (of 1 mL lysate, each) were used to infect one well each (without pooling). Methylcellulose overlay was added 4 h post infection. For the wildtype, P3 plaques y1, y3 and y5 gave similar numbers of red and green plaques, and both higher than yellow plaques. Plaque P3 y2 had a majority of red and some yellow and green plaques. Only in P3 y4 the number of yellow plaques exceeded number of green and red (FIG. 7 a). There were no non-fluorescent plaques.

For the CR19-derived plaques, four of the five isolates gave higher numbers of mono-fluorescent than yellow plaques (FIG. 7 b). Only for one isolate (P3 y1) infection resulted in a greater number of yellow than green and no red signals. This isolate was further investigated by infection with P3 y1 and with escalating dilutions into a 6-well plate (independent P4 in FIG. 7 b). Methylcellulose overlay was applied 5 h post infection. 3 days post-infection mainly yellow and mono-fluorescent green plaques were visible. The repeated presence of yellow plaques also under methylcellulose suggests that a true dual (red and green) expression of reporters has been established. The observation that red fluorescence was reduced when compared to previous expression levels and to green fluorescence is consistent with potential promoter interference or a genetic defect in the P11 promoter. Non-fluorescent (reversion to parental genotype) were not detected.

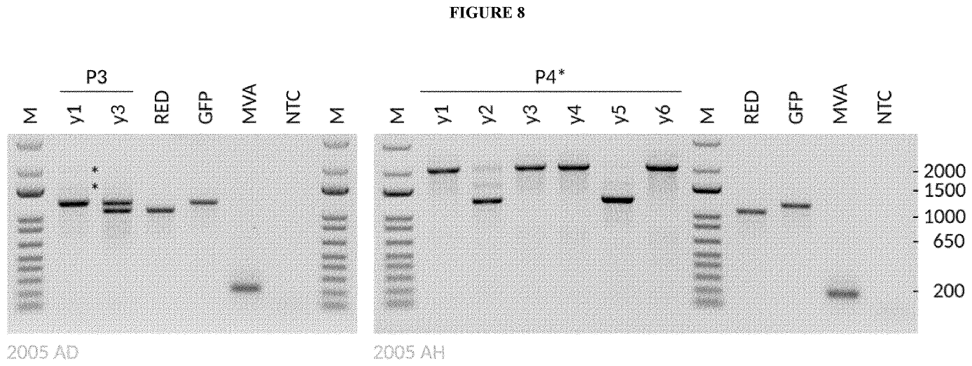

A total of six plaques were isolated from an independent infection with yellow plaque P3 y1 of CR19 for analysis of the genotype. Total DNA was recovered from 4 μL of the lysate of a plaque by adding 1 μL of QuickExtract™ DNA Extraction Solution 1.0 (Cambio, UK) and heating to 65 °C for 10 min and 98 °C for 5 min. 5 μL therefrom was used in a PCR reaction with final volume of 25 μL. PCR was performed with 35 cycles of 20 s at 94 °C, 20 s at 55 °C and 90s at 72 °C. PCR primers (SEQ ID 5 and SEQ ID 6) were chosen to amplify the complete insertion cassette including portions of the recombination flanks so that contamination by wildtype and due to partial deletions can be observed if present. This PCR that spans DS IV revealed a single amplicon that was higher than expected (approx. 2 kb instead of 1099 bp or 1231 bp) for 4 of the isolates and the expected 1099 bp for 4 PT-RED in two isolates (FIG. 8). However, both of the latter isolates also gave weak signals of approx. 2 kb and 1.5 kb.

The ancestral isolate P3 y1 gave a major amplification product at the same height as the p45T-GFP control and very faint signals for an intermediate and large insert. The isolate P3 y3 gave signals consistent with amplification of both, a GFP and mCherry cassette. None of the isolates gave amplicons of smaller sizes indicating that no revertants to an empty DS IV emerged.

A 17 μL aliquot of the PCR amplification product of the yellow isolate with the single approx. 2 kb band was purified with the QIAquick Gel Extraction kit and sequenced with the two amplification primers (LGC Genomics GmbH). The sequence confirmed presence of a large insert in DS IV that is capable of expression of both of the reporters.

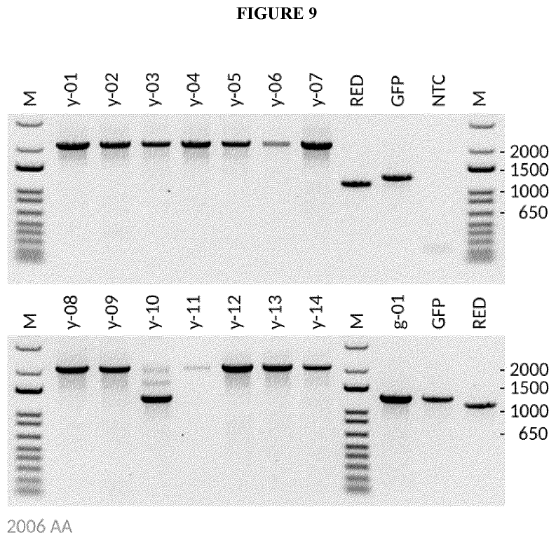

Both CR19 isolates with the mixed amplicons P4 y2 and P4 y5 and the isolate with the large insertion P4 y1 were infected into monolayers of a 6-well plates. The plate was analysed 2 days post infection and gave only yellow plaques for the isolate with the large insertion (y1). The other plaques with the dominant 4 PT-RED amplicon (y2 and y5) had a majority of yellow plaques (28 and 25, respectively) and fewer green plaques (6 and 15, respectively). Seven yellow P5 plaques each were isolated from P4 y1 (termed P5 y01-y07) and P4 y2 (y08-y14). One green plaque was picked of P4 y2 (now g1). 13 of the 14 yellow isolates gave a single large amplification product without apparent variation among the different preparations. The single exception showed a dominant band at the level of the GFP signal, a minor band for the large (2 kb) amplification product and another minor band in between. This middle band may be an intermediate from monofluorescence towards the yellow phenotype or away from the yellow phenotype towards monofluorescence. We tried to isolate and sequence this middle band but could not recover an amplification product suitable for sequencing. In a second production of the y2 virus isolate PCR gave only the smaller sized band for the 45T-GFP insert, supporting the hypothesis that the intermediate band is only transitory in a fast homology-directed copy pathway (FIG. 9).

It was next investigated whether a test of a yellow isolate in a comet assay will not yield any separation of fluorescent signals. First, virus in a yellow plaque from P4 y1 (FIG. 7) was expanded on a monolayer of CR.pIX cells in a 6-well plate and allowed to replicate without methylcellulose overlay. The culture was fully infected after 2 days with most cells in clear CPE but still attached. All cells appeared to express both reporters. A cryogenic lysate was generated and 2.15e8 TCID50/mL determined in a titration. The preparation was diluted for infection of monolayers in 6-well plates with 100 to 10 PFUs for subsequent methylcellulose overlay and comet assay.

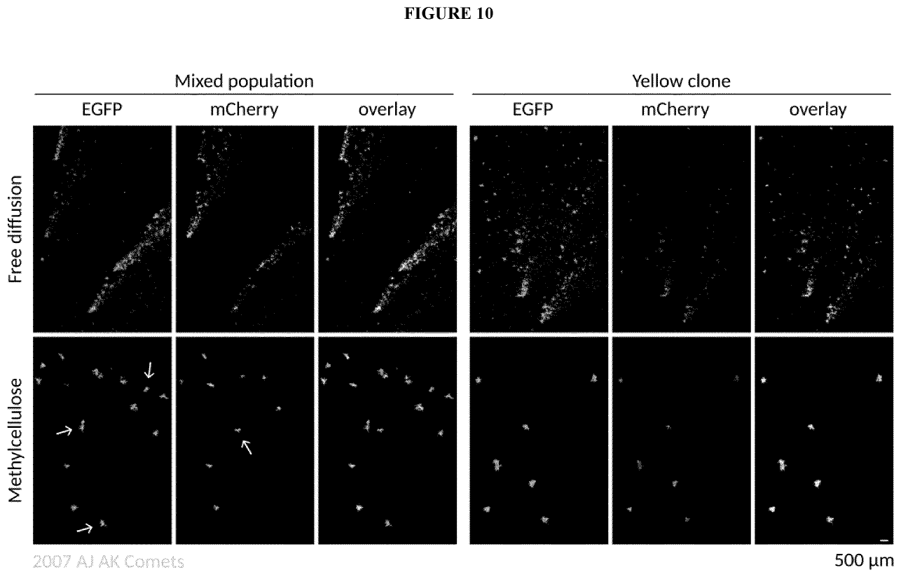

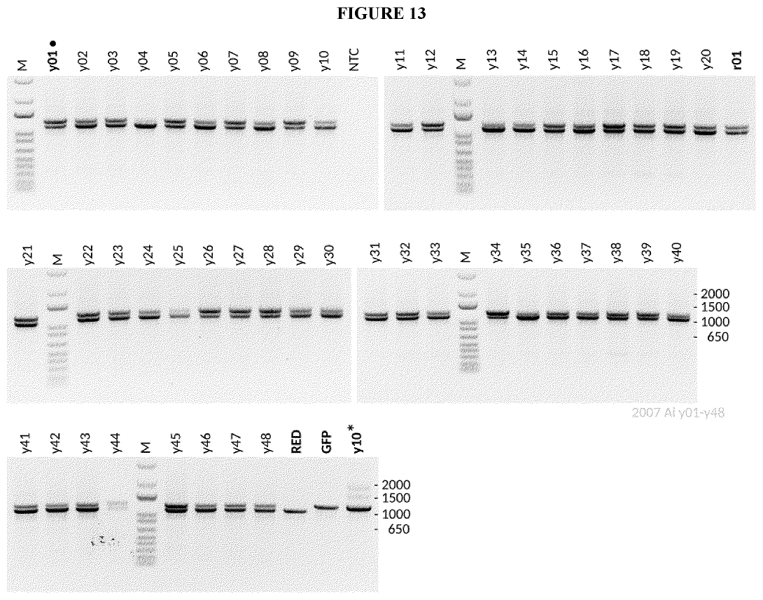

For comparison, preparation P2 y1 of FIG. 13 was used in a parallel infection with expectation that red, green and yellow (coinfection) plaques are visible under methylcellulose and that comets that separate into red and green tails. As shown in FIG. 10, expectations for both preparations under both conditions (free and impaired diffusion) were fullfilled. This result again confirms that coinfection with different viruses is a frequent event that can be quantified, and that the single truly yellow virus can has a different and unique phenotype.

### Example 4

Consecutive Insertion of mCherry into EGFP-Expressing MVA-CR19: Confirmation of Copy-Correction at the Terminal Positions

To complement the previous two studies, and to address the formal possibility that the pre-exisiting structure of the insert in DS IV may influence the outcome, recombination of the mCherry shuttle plasmid into an already GFP-containing virus was next performed. Here, it was also asked whether the unusual recombination into one larger dual expression cassette can be again observed. This larger recombination is unusual because only one flank for recombination is provided, not the corresponding distal second flank.

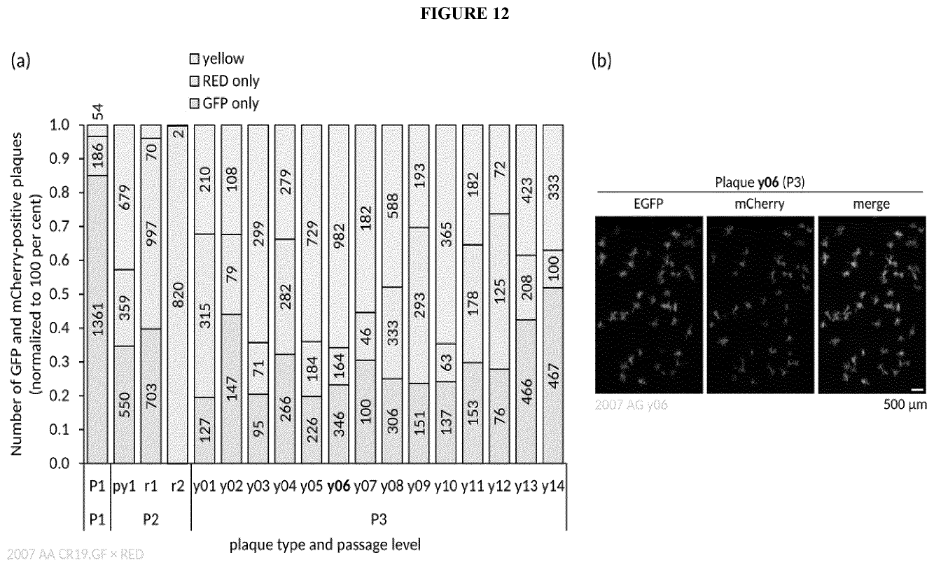

Two wells of a 6-well plate were infected with an MOI of 0.01. One well was transfected with p4PT-RED with Effectene (Qiagen) according to the manufacturer's instructions after 90 minutes, the other well was left uninfected as reference. A lysate was prepared after 2 days of the infected/transfected reaction and applied onto fresh cell monolayers in a 6-well plate at dilutions from 500-fold to 10\hat{ }6-fold. This P1 plate was scanned after 3 days, and 7 red and 36 yellow plaques were picked (out of a total of 1601 obtained plaques, FIG. 12).

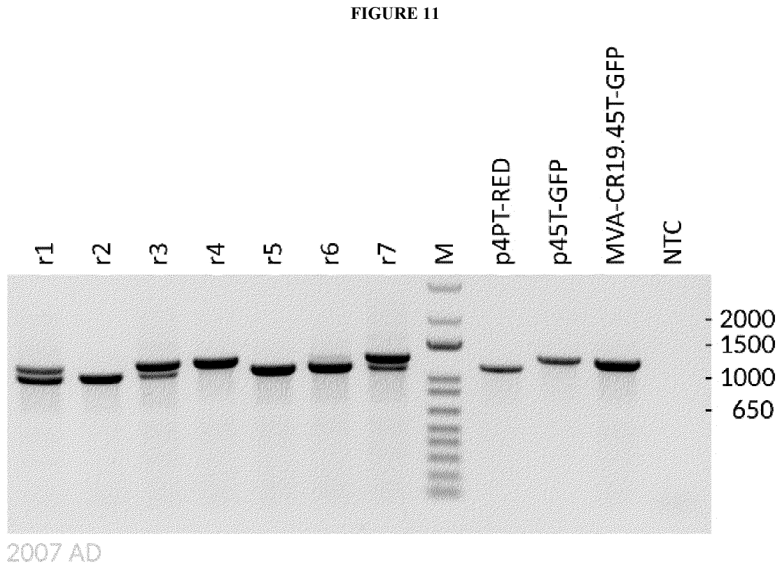

The 7 red P1 plaques are an indication of insert replacement (not simply insertion) and were tested by PCR against DS IV as described previously (FIG. 11). Positive controls were shuttle plasmids p4PT-RED and p45T-GFP and genomic DNA of the parental virus MVA-CR19.45T-GFP. Expected amplicons are 1099 bp for DS IV containing mCherry, 1231 bp for DS IV containing GFP, approx. 2000 bp for a combination of both reporter genes, and 220 bp for an empty (wildtype) DS IV (with primers SEQ ID 5 and 6).

There were no empty DS IV signals. Within one passage, plaques r2 and r5 already were R-R only. A simultaneous insertion of inserts into both termini (that are separated by more than 170 kb) is unlikely at the observed frequency of red signals from a parental green virus. The observation is more consistent with a mechanism by which both termini are juxtaposed and differences are corrected towards identical copies. This alignment or homology error correction appears to occur rapidly within very few virus generations. Double-bands in other lanes may transitorily events within a homogeneous preparation (R-G, G-R). However, the previous results strongly suggest a mixed population of new R-R and parental G-G viruses. Plaque r4 may have been a mispicked G-G parental virus. Erroneous isolation of unrelated viruses in neighbouring plaques is a common complication in plaque purification procedures and an indication that improved selection and purification methods for recombinant NCLDV vectors are highly relevant (see also FIG. 10).

Five plaques each of the yellow plaques of PI were pooled. In pools py1-py3 those were pooled that gave green and red fluorescence with similar intensities and good overlap (favorites for true R-G or G-R genotype).

Plaques r1 and r2, and pool py1 were used to infect a 6-well plate at dilutions of 0.05 and 0.2 (plaques) and 0.01 and 0.05 (pool), respectively. Methylcellulose was applied after 4 h. The wells infected with r1 resulted in mono-fluorescent red and green P2 plaques, consistent with the PCR result that again suggested presence of two distinct genotypes. A mixed genotype (R-G, G-R) would be expected to yield predominately yellow plaques. The wells infected with r2 gave only P2 red plaques, again consistent with the PCR result. The wells infected with pool py1 gave green, red and yellow P2 plaques. Out of the well with the 0.01 dilution, 14 P2 yellow plaques that appeared to be isolated from potentially contaminating neighbouring plaques were picked, singly resuspended in 1 mL medium and sonified. One well each of 6-well plates were infected at a 0.02 dilution with the isolated plaques to yield P3 y1-y14. Methylcellulose was applied after 4 h.

All 14 wells of the plates gave mixed populations of green, red and yellow plaques (FIG. 12). Pools py2 and py3 of the previous P1 plate were sonified and inoculated into fresh 6-well plates at dilutions 0.015, 0.01 and 0.005. Methylcellulose was applied after 3 h. A vast majority of mono-fluorescent red or green P2 plaques were observed 2 days post infection. 22 yellow plaques each from pools py2 and py3 were picked into 20 μL medium each, resulting in a total of 48 P2 isolates that were tested by PCR against DS IV. Controls were shuttle plasmids p4PT-RED and p45T-GFP, and genomic DNA of a P2 r1 with mixed genotype and an incompletely matured y10.

PCR resulted for all isolates in a double band typical for separate GFP and mCherry cassettes (FIG. 13), and none gave a band that suggested either an empty DS IV or dual-reporter recombination.

In summary, plaques with mixed (green and red) colors were extremely infrequent in our experiments. Most plaques that gave an appearance of dual fluorescence were shown to be superimposed plaques of different colors at higher magnification. PCR reactions with plaque-purified viruses out of these plaques usually separated into either R-R or G-G viruses. Possibly only a single exception of true dual fluorescence was observed. However, these viruses did not show two distinct mCherry and GFP amplicons but rather a novel larger amplicon indicative of recombiniation towards an RG-GR genotype. PCR after recombination suggested that in the mixed populations of green and red plaques conversion to a single fluorescence happens rapidly (within one passage) and is stable as no deletions towards empty or collapsing DS IV were encountered.

### Example 5

### Advantageous Expression

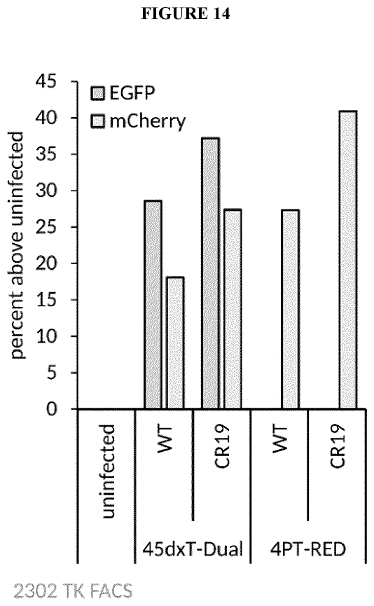

Hence, as shown above, one advantage of having diploid expression cassettes localized to the terminal inverted repeats of a poxvirus is demonstrated by a potential error correction of the sequences towards maintenance of identical copies. It was next searched for another advantage, effects of doubling of the gene dosis on expression strength. Doubling of a gene does not automatically lead to improved gene expression as promoters may interfere. However, as shown in FIG. 14, for two different reporter genes (EGFP and mCherry) and in two different expression designs (dual expression driven by mH5dx and P11) and single expression (P11 only) higher signal frequencies with MVA-CR19 compared to wildtype was observed.

The data in FIG. 14 was obtained by infection of 1.2× 107 CRp.IX suspension cells with MVA.45dxT-Dual, MVA-CR19.45dxT-Dual, MVA-A3.4 PT-RED or MVA-CR19.4 PT-RED at MOI of 0.1. After 24 h, cells were collected by centrifugation with 200×g for 5 min at 4 °C and resuspended in 3 ml PBS. Fluorescence intensity was measured in 100 μl samples with the MACSQuant® Analyzer 10 Flow Cytometer. At least 23000 events are contained in each measurement. Identical gates were applied for wildtype and CR19.

### REFERENCES

Chakrabarti S, Sisler J R, Moss B (1997) Compact, synthetic, vaccinia virus early/late promoter for protein expression. BioTechniques 23:1094-1097
Dobson B M, Tscharke D C (2015) Redundancy complicates the definition of essential genes for vaccinia virus. J Gen Virol 96:3326-3337. https://doi.org/10.1099/jgv.0.000266.
Jordan I, Horn D, Thiele K, Haag L, Fiddeke K, Sandig V (2019) A Deleted Deletion Site in a New Vector Strain and Exceptional Genomic Stability of Plaque-Purified Modified Vaccinia Ankara (MVA). Virol Sin. https://doi.org/10.1007/s12250-019-00176-3.
Jordan I, Vos A, Beilfuss S, Neubert A, Breul S, Sandig V (2009) An avian cell line designed for production of highly attenuated viruses. Vaccine 27:748-756. https://doi.org/10.1016/j.vaccine.2008.11.066
Kremer M, Volz A, Kreijtz J H C M, Fux R, Lehmann M H, Sutter G (2012) Easy and efficient protocols for working with recombinant vaccinia virus MVA. Methods Mol Biol Clifton NJ 890:59-92. https://doi.org/10.1007/978-1-61779-876-4_4
Senkevich T G, Bruno D, Martens C, Porcella S F, Wolf Y I, Moss B (2015) Mapping vaccinia virus DNA replication origins at nucleotide level by deep sequencing. Proc Natl Acad Sci US A 112:10908-10913. https://doi.org/10.1073/pnas. 1514809112

### INFORMAL SEQUENCE LISTING

SEQ ID 1:
ttacatgtgactgtgttcagggtatag
SEQ ID 2:
catagctagccgtacatccacatct
SEQ ID 3:
ttagcggccgcgtacggctttagaaatgag
SEQ ID 4:
ttcacgtagtgggtctgaactgggcac
SEQ ID 5:
gtgctataacgcgactatctag
SEQ ID 6:
tgttggtagttcttccgtgg
SEQ ID 7:
tttggtaatggtttctcatgtgg
SEQ ID 8:
gacatttagtttgagtgttcctg
SEQ ID 9:
ctttcgcagcataagtagtatgtc
SEQ ID 10:
cattaccgcttcattcttatattc

1. A nucleocytoplasmic large DNA virus (NCLDV) containing a polynucleotide comprising an identical nucleotide sequence of interest within each Inverted Terminal Repeat (ITR).

2. The NCLDV of claim 1, wherein the identical nucleotide sequences of interest are comprised at identical positions within the Inverted Terminal Repeats (ITRs).

3. The NCLDV of claim 2, wherein the identical nucleotide sequences of interest are comprised between non-essential viral genes or replace one or more non-essential viral genes.

4. The NCLDV of claim 1, wherein the NCLDV is a virus of the Poxviridae, Asfarviridae, or Iridoviridae family.

5. The NCLDV of claim 4, wherein the virus of the Poxviridae family is selected from the group consisting of a vaccinia virus, an avipoxvirus, and a parapoxvirus.

6. The NCLDV of claim 5, wherein
the vaccinia virus is an attenuated vaccinia virus selected from the group consisting of a Modified Vaccinia Ankara (MVA) virus, an MVA related virus, a vaccinia virus Dairen-I, and a vaccinia virus strain NYVAC,
the avipoxvirus is selected from the group consisting of a canarypox virus and a fowlpox virus, or
the parapoxvirus is an ORF virus.

7. The NCLDV of claim 6, wherein the MVA virus comprises one of the identical nucleotide sequences of interest in the right Inverted Terminal Repeat (ITR) and the other one of the identical nucleotide sequences of interest in the left Inverted Terminal Repeat (ITR).

8. The NCLDV of claim 6, wherein the MVA related virus contains a polynucleotide comprising the identical nucleotide sequences of interest in each deletion site IV.

9-10. (canceled)

11. The NCLDV of claim 1, wherein the nucleotide sequences of interest are stably maintained by/within the NCLDV.

12. (canceled)

13. The NCLDV of claim 1, wherein the nucleotide sequences of interest are selected from the group consisting of a sequence coding for an antigen, particularly an epitope of an antigen, a diagnostic compound, and a therapeutic compound.

14. (canceled)

15. A method for producing a nucleocytoplasmic large DNA virus (NCLDV) containing a polynucleotide comprising an identical nucleotide sequence of interest within each Inverted Terminal Repeat (ITR) comprising the steps of:
(i) transfecting a cell line infected with a nucleocytoplasmic large DNA virus (NCLDV) with a nucleotide sequence of interest, wherein the nucleotide sequence of interest is flanked by regions capable of homologous recombination within the Inverted Terminal Repeats (ITRs) of the NCLDV, thereby obtaining a NCLDV containing a polynucleotide comprising a nucleotide sequence of interest within one of the ITRs, and
(ii) culturing the NCLDV virus obtained in (i) under conditions allowing homologous repair between the Inverted Terminal Repeats (ITRs), thereby obtaining a NCLDV containing a polynucleotide comprising an identical nucleotide sequence of interest within each Inverted Terminal Repeat (ITR).

16. The method of claim 15, wherein the cell line is selected from the group consisting of an avian cell line, preferably a chicken retina cell line AGE1.CR.PIX, a DF-1 chicken fibroblast cell line, or a duck embryonic stem cell line EB66, a mammalian cell line, preferably a baby hamster kidney (BHK) cell line or a human embryonal kidney 293 (HEK293) cell line, an arthropod cell line, preferably a Sf9 cell line, and a piscine cell line, preferably a SAF-1 cell line.

17. The method of claim 15, wherein the NCLDV is a virus of the Poxviridae, Asfarviridae, or Iridoviridae family.

18. The method of claim 17, wherein the virus of the Poxviridae family is selected from the group consisting of a vaccinia virus, an avipoxvirus, and a parapoxvirus.

19. The method of claim 18, wherein
the vaccinia virus is an attenuated vaccinia virus selected from the group consisting of a Modified Vaccinia Ankara (MVA) virus, an MVA related virus, a vaccinia virus Dairen-I, a vaccinia virus strain NYVAC,
the avipoxvirus is selected from the group consisting of a canarypox virus and a fowlpox virus, or
the parapoxvirus is an ORF virus.

20. The method of claim 15, wherein the nucleotide sequences of interest are selected from the group consisting of a sequence coding for an antigen, particularly an epitope of an antigen, a diagnostic compound, and a therapeutic compound.

21-22. (canceled)

23. A method for propagating a nucleocytoplasmic large DNA virus (NCLDV) of claim 1 comprising the step of: culturing the NCLDV in a cell line.

24. The method of claim 23, wherein the cell line is selected from the group consisting of an avian cell line, preferably a chicken retina cell line AGE1.CR.PIX, a DF-1 chicken fibroblast cell line, or a duck embryonic stem cell line EB66, a mammalian cell line, preferably a baby hamster kidney (BHK) cell line or a human embryonal kidney 293 (HEK293) cell line, an arthropod cell line, preferably a Sf9 cell line, and a piscine cell line, preferably a SAF-1 cell line.

25. A pharmaceutical composition comprising a nucleocytoplasmic large DNA virus (NCLDV) of claim 1.

26-28. (canceled)

29. A method for treating a patient, comprising the step of administering a therapeutically effective amount of a nucleocytoplasmic large DNA virus (NCLDV) of claim 1 or a pharmaceutical composition comprising the NCLDV to a patient in need thereof.
